(19) United States
(12) Patent Application Publication
(10) Pub. No.: US 2024/0093206 A1
(43) Pub. Date: Mar. 21, 2024

(54) SYSTEM OF STABLE GENE EXPRESSION IN CELL LINES AND METHODS OF MAKING AND USING THE SAME

(71) Applicant: 490 BioTech, Inc., Knoxville, TN (US)

(72) Inventors: Daniel Close, Knoxville, TN (US); Steven Ripp, Knoxville, TN (US); Gary Sayler, Blaine, TN (US)

(21) Appl. No.: 18/282,320

(22) PCT Filed: Mar. 15, 2022
(86) PCT No.: PCT/US22/20370

§ 371 Date: Sep. 15, 2023

(51) Int. Cl. C12N 15/52; C12N 5/071; C12N 9/00; C12N 9/02; C12N 9/10; C12N 15/85

(52) U.S. Cl. CPC C12N 15/52; C12N 5/0686; C12N 9/0008; C12N 9/0071; C12N 9/1029; C12N 9/93; C12N 15/85; C12Y 102/0105; C12Y 114/14003; C12Y 203/01; C12Y 602/01019

(57) ABSTRACT

A system for stable expression of gene pathways in cell lines, methods of making cell lines with stable expression of gene pathways, and methods of using the same are disclosed herein. The system comprises a nucleic acid construct configured to encode at least two genes of a multigene pathway in a cell. The nucleic acid construct comprises a plurality of nucleic acid sequences, wherein the plurality of nucleic acid sequences comprises: a first nucleic acid sequence encoding at least one gene of the multigene pathway; a first protease recognition nucleic acid sequence encoding a protease recognition site; a first linker nucleic acid sequence encoding a linker region, wherein the linker region comprises a viral 2A peptide; and a second nucleic acid sequence encoding at least one gene of the multigene pathway.

### CROSS-REFERENCE TO RELATED APPLICATION

This application cites the priority of currently pending U.S. 63/161,059 filed Mar. 15, 2021. U.S. 63/161,059 is incorporated herein by reference in its entirety.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under grant 1R43ES026269 from the National Institute of Environmental Health Sciences, an institute of the National Institutes of Health. The government has certain rights in this invention. In this context “government” refers to the government of the United States of America.

### REFERENCE TO SEQUENCE LISTING

The material in the accompanying sequence listing is hereby incorporated by reference in its entirety. The accompanying file, named 218101_401011_Sequence_Listing_for_PCT_ST25.txt, is 144 KB, was created on Mar. 14, 2022, and was electronically submitted via EFS-Web on Mar. 15, 2022.

### FIELD OF THE INVENTION

The disclosed technology is directed towards a system for stable expression of gene pathways in cell lines, methods of making cell lines that stably express gene pathways, and methods of using the same.

### BACKGROUND OF THE INVENTION

The background discussion is included to explain the context of the present disclosure. Any information included in this section is not to be taken as an admission that any of the material referred to was published, known, or part of the common general knowledge in any country as of the priority date of any of the claims.

There is a clear need for low cost, high-throughput, non-invasive cellular monitoring methods in biological fields such as drug development, toxicology, environmental monitoring, and basic research. Bioluminescence, the production of light from a living cell, would be an ideal detection modality for these applications; however, bioluminescence has not been employed due to numerous hinderances. Such hindrances include, for example, a limitation to only single time points, a requirement for expensive externally-applied reagents to function across a limited time span, and an inability to be exogenously expressed at temperatures relevant for most applications. Thus, there is an unmet need for adaption of a bioluminescent system capable of stable continuous and/or autonomous function.

### SUMMARY OF THE INVENTION

In one aspect, the present invention provides a nucleic acid construct configured to encode at least two genes of a multigene pathway in a cell. In embodiments, the nucleic acid construct comprises a plurality of nucleic acid sequences, wherein the plurality of nucleic acid sequences comprises: a first nucleic acid sequence encoding at least one gene of the multigene pathway; a first protease recognition nucleic acid sequence encoding a protease recognition site; a first linker nucleic acid sequence encoding a linker region, wherein the linker region comprises a viral 2A peptide; and a second nucleic acid sequence encoding at least one gene of the multigene pathway. In certain embodiments, the first nucleic acid sequence and the second nucleic acid sequence are joined via the first linker nucleic acid sequence, and the first protease recognition nucleic acid sequence is located between the first nucleic acid sequence and the first linker nucleic acid sequence. One or more of the plurality of nucleic acid sequences are adjacent and bonded to one another via a phosphodiester bond, a phosphorothionate bond, or a combination thereof.

In embodiments, the multigene pathway is thermostable at a cell culture relevant temperature.

In the various embodiments, the first nucleic acid sequence comprises a first luciferin/luciferase nucleic acid sequence, the second nucleic acid sequence comprises a second luciferin/luciferase nucleic acid sequence, and the multigene pathway comprises a luciferin/luciferase pathway. The first luciferin/luciferase nucleic acid sequence and the second luciferin/luciferase nucleic acid sequence can be configured to encode different genes of the luciferin/luciferase pathway. The plurality of nucleic acid sequences can further comprise a third nucleic acid sequence that encodes one or more of: an oxidoreductase gene, a second protease recognition nucleic acid sequence encoding a second protease recognition site, and a second linker nucleic acid sequence encoding a second linker region, wherein the second linker region comprises a viral 2A peptide. In embodiments, the second nucleic acid sequence and the third nucleic acid sequence are joined via the second linker nucleic acid sequence, and the second protease recognition nucleic acid sequence is located between the second nucleic acid sequence and the second linker nucleic acid sequence. In certain embodiments, the oxidoreductase gene comprises frp. The luciferin/luciferase pathway can comprise a bacterial luciferin/luciferase pathway, a fungal luciferin/luciferase pathway, or a combination thereof. In embodiments, the first luciferin/luciferase nucleic acid sequence or the second luciferin/luciferase nucleic acid sequence encode for one or more of: luxC, luxD, luxA, luxB, luxE, luxF, luxG, luxH, luxI, luxR, luxY, or frp. The first luciferin/luciferase nucleic acid sequence or the second luciferin/luciferase nucleic acid sequence can encode for one or more genes involved in synthesis of caffeic acid.

In embodiments, the one or more genes involved in synthesis of caffeic acid comprise: a tyrosine ammonia lyase, two 4-hydroxyphenylacetate 3-monooxygenase components, a 4′-phosphopantetheinyl transferase, or a combination thereof.

In one embodiment, the first luciferin/luciferase nucleic acid sequence or the second luciferin/luciferase nucleic acid sequence encode for one of more of: luz, H3H, or HipS.

In certain embodiments, the nucleic acid construct comprises at least six luciferin/luciferase nucleic acid sequences, wherein each of the at least six luciferin/luciferase nucleic acid sequences encodes for a different gene of the luciferin/luciferase pathway. The different genes of the luciferin/luciferase pathway comprise luxC, luxD, luxA, luxB, luxE, and frp.

The first luciferin/luciferase nucleic acid sequence or the second luciferin/luciferase nucleic acid sequence can be at least about 90% identical to SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, or SEQ ID NO: 47. The first luciferin/luciferase nucleic acid sequence or the second luciferin/luciferase nucleic acid sequence can encode for an amino acid sequence that is at least about 90% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15.

In embodiments, at least one of the plurality of nucleic acid sequences encodes a gene for a luciferase enzyme. At least one of the plurality of nucleic acid sequences can encode a gene for a protein required for luciferin substrate production. The protease recognition site can comprise a recognition site for furin. The protease recognition nucleic acid sequence may encode an amino acid sequence comprising R-X-X-R. The protease recognition nucleic acid sequence may encode an amino acid sequence comprising R-K-R-R.

In embodiments, the viral 2A peptides comprise T2a, E2a, F2a, P2a, Pa2a, FMDV2a, or a combination thereof. The linker nucleic acid sequence can encode an amino acid sequence comprising at least 90% identity to SEQ ID NO: 28, SEQ ID NO: 29, SEQ ID NO: 30, SEQ ID NO: 31, SEQ ID NO: 32, or a combination thereof. The linker nucleic acid sequence can comprise at least 90% identity to SEQ ID NO: 60, SEQ ID NO: 61, SEQ ID NO: 62, SEQ ID NO: 63, SEQ ID NO: 64, or a combination thereof.

The nucleic acid construct can comprise at least one spacer region between the nucleic acid sequences. In embodiments, the at least one spacer region comprises a plurality of nucleotides capable of: targeting mRNA or protein products to specific locations within the cell or extracellularly; increasing the distance between the nucleic acid sequences; imparting structures that modify the efficiency of a protease or a ribosome at the DNA, RNA, or polypeptide level; encoding at least one flexible protein region to modify a functionality or efficiency of the linker region; or a combination thereof.

The nucleic acid construct can further comprise a promoter, an enhancer, an operator, or other element capable of initiating or regulating transcription or translation of the nucleic acid sequences.

In embodiments, the nucleic acid construct can further comprise at least one stop codon, a poly-A sequence, a terminator, or other element capable of stopping transcription or translation of one or more of the nucleic acid sequences.

In another aspect, the present invention provides a vector comprising any one or more of the nucleic acid constructs disclosed herein.

Another aspect provides for a cell that comprises the above-referenced vector.

In yet another aspect, the present invention provides for a method of producing bioluminescence in a cell line. In embodiments, the method comprises introducing any of the nucleic acid constructs disclosed herein into a plurality of cells to form a plurality of transfected cells, expressing the nucleic acid construct in the plurality of transfected cells, maintaining the plurality of transfected cells in a culture media and at a cell culture relevant temperature, and forming an autonomously bioluminescent cell line by isolating one or more of the plurality of transfected cells.

In embodiments, cell culture relevant temperature comprises a temperature of at least about 4° C.

In another aspect, the present disclosure provides for a system for expression of bioluminescence in cells. In certain embodiments, the system comprises a cell line comprising any of the nucleic acid constructs disclosed herein, the nucleic acid construct having a luciferase/luciferin pathway functional at temperatures used in generating cell cultures, growing cell cultures, maintaining cell cultures, or a combination thereof. The temperatures used in generating cell cultures, growing cell cultures, maintaining cell cultures, or a combination thereof can comprise temperatures of greater than about 4° C. In embodiments, the temperatures used in generating cell lines, growing cell cultures, maintaining cell cultures, or a combination thereof comprise temperatures up to about 42° C. The temperatures used in generating cell cultures, growing cell cultures, maintaining cell cultures, or a combination thereof can comprise temperatures of about 37° C.

In embodiments, the cell line comprises eukaryotic cells.

In another aspect, the present disclosure provides for a system for co-expression of at least two functional luciferase/luciferin pathway genes in a cell. In embodiments, the system comprises: a first luciferase/luciferin pathway gene, wherein the first luciferase/luciferin pathway gene is transfected into a cell; and a second luciferase/luciferin pathway gene transfected into the cell, wherein the first and second luciferase/luciferin pathway genes are disposed within a single nucleic acid construct and form a luciferase/luciferin pathway that is capable of autonomously producing bioluminescence in the cell at cell culture relevant temperatures.

The cell culture relevant temperature can comprise a temperature of at least about 4° C. In embodiments, the cell culture relevant temperatures comprise temperatures up to about 42° C. The cell culture relevant temperatures can comprise temperatures of about 37° C. In certain embodiments, the cell line comprises eukaryotic cells.

Another aspect of the present disclosure includes a method of non-invasive cellular monitoring. In embodiments, the method comprises providing at least one cell producing bioluminescence, the cell having been transfected with any of the various nucleic acid constructs disclosed herein; and monitoring the bioluminescence of the cell. The bioluminescence may be detectable at multiple time points and in real-time In embodiments, the bioluminescence is detectable in the absence of an exogenous luminescent stimulator.

In yet another aspect, the present invention provides for a nucleic acid cassette comprising components in the following structure, oriented in a 5′ to 3′ direction: A-p-B-C(n), wherein: “A” comprises a nucleic acid sequence encoding at least one gene of a luciferase/luciferin pathway; “p” comprises a nucleic acid sequence encoding a protease recognition site; “B” comprises a nucleic acid sequence encoding a 2A peptide; “C” comprises a nucleic acid sequence encoding at least one gene of a luciferase/luciferin pathway; and “n” is the number of repetitions of the “-p-B-C” portion of the nucleic acid cassette. In embodiments, “-” comprises a phosphodiester bond, a phosphorothioate bond, or a combination thereof. In embodiments, “n” comprises a first repetition and at least one additional repetition, and wherein B, C, or both in the first repetition are not identical to B, C, or both, respectively, in the at least one additional repetition. In certain embodiments, “n” is 1, 2, 3, 4, 5, 6, 7, 8, 9, or 10. In one embodiment, “n” is at least 10.

The nucleic acid cassette can comprise a localization signal or an excretion signal for targeted expression within a cell or trafficking to outside of a cell. In embodiments, the nucleic acid cassette comprises at least one sequence tag for isolation, identification, visualization, or a combination thereof.

The nucleic acid cassette can comprise an element configured to initiate, enhance, regulate, or stop transcription or translation of A, p, B, C, or a combination thereof.

In another aspect, the present invention provides for a vector comprising the nucleic acid cassette as described herein. The vector can be an expression vector.

In a further aspect, the present invention provides a kit for producing a genetically engineered cell having autonomous luminescence, the kit comprising a vector comprising any nucleic acid construct(s) disclosed herein.

In still further aspects, the present invention provides a method for producing a genetically engineered cell having autonomous luminescence, the method including transfecting a cell with a vector comprising any of the nucleic acid constructs disclosed herein.

In yet other aspects, the present invention provides a method of real-time monitoring of cell population size of a genetically engineered cell having autonomous luminescence, the method including transfecting a cell with a vector comprising any of the nucleic acid constructs disclosed herein to produce the genetically engineered cell having autonomous luminescence; measuring a luminescent signal emitted from the genetically engineered cell having autonomous luminescence; and assessing the cell population size of the genetically engineered cell having autonomous luminescence based on the measured luminescent signal.

In still further aspects, the present invention provides a method of real-time monitoring of cell viability of a genetically engineered cell having autonomous luminescence, the method including transfecting a cell with a vector comprising any of the nucleic acid constructs disclosed herein to produce the genetically engineered cell having autonomous luminescence; measuring a luminescent signal emitted from the genetically engineered cell having autonomous luminescence; and assessing the cell viability of the genetically engineered cell having autonomous luminescence based on the measured luminescent signal.

Other objects and advantages of this invention will become readily apparent from the ensuing description.

### BRIEF DESCRIPTION OF THE FIGURES

FIG. 1 illustrates an overview of the system according to one embodiment. Multiple open readings frames (ORFs) are connected by intervening protease recognition sequences and 2A linkers. This architecture can be repeated as many times as needed to encode the open reading frames necessary for the desired functionality.

FIG. 2 shows the functionality of the FIG. 1 system. A) The 2A elements allow a single encoded sequence to be transcribed and translated into B) individual proteins with artifactual amino acid residues from the protease recognition sites and 2A linkers attached. C) Endogenous proteases remove the artifactual amino acid residues, resulting in individual proteins that more closely match their native amino acid identity.

FIG. 3 shows that linking luciferin/luciferase pathway genes using 2A elements results in decreased light production compared to expression without the artifactual amino acids that remain following translation of individual proteins.

FIG. 4 shows that incorporation of furin recognition sites upstream of viral 2A linkers between codon optimized bacterial luciferase genes in HEK293 cells significantly improves bioluminescent production at 37° C. Briefly, removal of 2A linker artifactual amino acids resulted in a 133 (±9) fold increase in light output compared to using only 2A linkers and retaining the artifactual amino acid sequences at the C-terminus of the luciferin/luciferase genes.

FIG. 5 shows that signal output remains steady following stable integration of bacterial luciferase genes without artifactual amino acid residues from 2A linkers in HEK293 cells. Cells were transfected with a version of the bacterial luciferase cassette designed to eliminate artifactual amino acids resulting from 2A element cleavage. Bioluminescent production was measured from the same lineage of cells at 1 and 16 passages (56 days apart) after stable expression was established. No significant difference in expression (p>0.01) was observed.

### DETAILED DESCRIPTION OF THE INVENTION

For a cell to autonomously produce a luminescent signal it must express genes for both the luciferase enzyme and the proteins required for substrate production, trafficking, and regeneration. These pathways may require co-expression of more than one gene. Modulation, or lack thereof, of the luminescent phenotype may require dependent or independent expressional control of individual luciferase or substrate processing genes, groups of luciferase or substrate processing genes, or the full pathway of luciferase and substrate processing genes. Co-expression may require genes to be linked to enable multiple proteins to be obtained from a single mRNA sequence.

Luminescent systems with known luciferin/luciferase pathways, such as bacterial luciferase or fungal luciferase, require expression of multiple genes to enable autonomous bioluminescent production. Efficient introduction of these multiple genes into naturally non-luminescent hosts requires them to be linked so more than one gene is incorporated into the genome at a time. The required linker regions can result in reduced functionality. In some cases, such as for bacterial luciferase, this significantly impairs functionality at 37° C., resulting in diminished light output under standard culture conditions. As a result, there have been no successful demonstrations of the stable generation of continuously or autonomously bioluminescent animal cells using any luminescent system with a known luciferin/luciferase pathway that functions efficiently at its optimal growth temperature

Embodiments as described herein confront this problem and are directed towards stable, multigene expression of luciferin/luciferase pathway genes for thermostable protein expression, allowing continuous or autonomous light production in the host.

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein can have the same meaning as commonly understood by one of ordinary skill in the art to which this disclosure belongs. Any methods and materials similar or equivalent to those described herein can also be used in the practice or testing of the present disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### Abbreviations and Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including,” and the like are used herein, the phrase “and without limitation” is understood to follow unless explicitly stated otherwise. Similarly, “an example,” “exemplary,” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising,” “including,” “having,” “involving” (and similarly “comprises,” “includes,” “has,” and “involves”) and the like are used interchangeably and have the same meaning. Specifically, each of the terms is defined consistent with the common United States patent law definition of “comprising,” is therefore interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it can modify that range by extending the boundaries above and below the numerical values set forth. In general, the term “about” is used herein to modify a numerical value above and below the stated value by a variance of 20 percent up or down (higher or lower).

The terms “bioluminescent,” “luminescent,” and similar phrases may be used interchangeably. Further, “autonomously bioluminescent,” “autonomously luminescent,” “autobioluminescence,” and similar phrases may be used interchangeably. A cell is autonomously bioluminescent, or has autobioluminescence, when it self-synthesizes all of the substrates required for luminescent signal production, e.g., through expression of the luciferase (lux) cassette. That is, the mechanism for producing bioluminescence (also referred to as a luminescent or bioluminescent signal) operates autonomously and in real-time to indicate cellular and molecular mechanisms coupled to bioluminescent signal output. Cells and methods of making and using cells having autobioluminescence are described in U.S. Pat. No. 7,300,792, which is incorporated by reference in its entirety.

The term “codon optimization” encompasses a strategy in which codons within a cloned gene—codons not generally used by the host cell translation system—are changed by mutagenesis, or any other suitable means, to the preferred codons of the host organism, without changing the amino acids of the synthesized protein.

The terms “encodes” and “encoding” refer to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system.

The term “expression” refers to the translation of a nucleic acid into a protein. Proteins may be expressed and remain intracellular, become a component of the cell surface membrane, or be secreted into the extracellular matrix or medium.

The term “lux cassette” refers to the bacterial luciferase (lux) gene cassette that comprises five genes: the luxC gene, the luxD gene, the luxA gene, the luxB gene, and the luxE gene. These five genes encode protein products that synergistically interact to generate bioluminescent light without the addition of an auxiliary substrate. Moreover, there is an additional gene, the flavin reductase gene (referred to as either “frp” or “F”), that functions as a flavin reductase to aid in cycling endogenous flavin mononucleotide into the FMNH2 co-substrate required for the aforementioned bioluminescence reaction. These genes may be referred to in shorthand notation. For example, when referring to all five genes of the lux cassette, the shorthand notation may be luxCDABE. When referring to only a subset of said genes, the shorthand notation may be luxAB, luxCDE, or any other combination. Shorthand notation may also be employed to refer to the flavin reductase gene. For example, when referring to the flavin reductase gene with the lux cassette, the shorthand notation may be either luxCDABEfrp or luxCDABEF. The luxC gene, the luxD gene, the luxA gene, the luxB gene, the luxE gene, and frp may each have a wild type sequence, a codon optimized sequence, and variations, derivations, and modifications thereof. Unless otherwise provided, references to the luxC gene, the luxD gene, the luxA gene, the luxB gene, the luxE gene, and frp encompass the wild type sequence and the codon optimized sequence and variations, derivations, and modifications thereof.

As used herein, the terms “polynucleotide” and “nucleic acid sequence” can be used interchangeably to refer to nucleotide polymers of DNA, RNA, or a fragment thereof. In embodiments, the terms “polynucleotide” and “nucleic acid sequence” comprise a synthetic polynucleotide. A polynucleotide may include methylated nucleotides.

As used herein, the terms “peptide,” “polypeptide,” and “protein” are used interchangeably herein. Unless otherwise clear from the context, the aforementioned terms can refer to a polymer having at least two amino acids linked through peptide bonds, non-liming examples of which comprise oligopeptides, protein fragments (such as functional domains), glycosylated derivatives, pegylated derivatives, fusion proteins, and the like.

As used herein, a “protease recognition site” is a contiguous sequence of amino acids connected by peptide bonds that contains a pair of amino acids which is connected by a peptide bond that is hydrolyzed by a particular protease. Optionally, a protease recognition site can include one or more amino acids on either side of the peptide bond to be hydrolyzed, to which the catalytic site of the protease also binds (Schecter and Berger, (1967) Biochem. Biophys. Res. Commun. 27: 157-62), or the recognition site and cleavage site on the protease substrate can be two different sites that are separated by one or more (e.g., two to four) amino acids.

The specific sequence of amino acids in the protease recognition site typically depends on the catalytic mechanism of the protease, which is defined by the nature of the functional group at the protease's active site. For example, trypsin hydrolyzes peptide bonds whose carbonyl function is donated by either a lysine or an arginine residue, regardless of the length or amino acid sequence of the polypeptide chain. Factor Xa, however, recognizes the specific sequence Ile-Glu-Gly-Arg (SEQ ID NO:19) and hydrolyzes peptide bonds on the C-terminal side of the Arg.

Thus, in various embodiments, a protease recognition site can comprise at least 2, 3, 4, 5, 6, 7, 8, 9, or 10 or more amino acids. Optionally, additional amino acids can be present at the N-terminus and/or C-terminus of the recognition site. A protease recognition site according to the invention also can be a variant of a recognition site of a known protease as long as it is recognized/cleaved by the protease.

Various preferred protease recognition sites include, but are not limited to, protease recognition sites for proteases from the serine protease family, for metalloproteases, the cysteine protease family, the aspartic acid protease family, and/or the glutamic acid protease family. In certain embodiments preferred serine proteases recognition sites include, but are not limited to, recognition sites for chymotrypsin-like proteases, subtilisin-like proteases, alpha/beta hydrolases, and/or signal peptidases. In certain embodiments, preferred metalloprotease recognition sites include, but are not limited to, recognition sites for metallocarboxypeptidases or metalloendopeptidases.

Protease recognition sites are well known to those of skill in the art. Recognition sites have been identified for essentially every known protease. Thus, for example, recognition sites (peptide substrates) for the caspases are described by Earnshaw et al. (1999) Annu. Rev. Biochem., 68: 383-424, which is incorporated herein by reference.

As used herein, the terms “open reading frame” (ORF), “transgene,” or “(trans)gene” are used interchangeably and can refer to a particular nucleic acid sequence encoding a polypeptide or a portion of a polypeptide to be expressed in a cell into which the nucleic acid sequence is inserted.

The term “read” refers to a DNA sequence of sufficient length (e.g., at least about 30 bp) that can be used to identify a larger sequence or region (e.g., that can be aligned and specifically assigned to a chromosome or genomic region or gene).

The term “sequence tag” is used to refer to a sequence read that has been specifically assigned (e.g., mapped) to a larger sequence (e.g., a reference genome by alignment).

The term “vector” can refer to nucleic acid molecules, usually double-stranded DNA, which may have inserted into it, such as within its backbone or coding region, another nucleic acid molecule (the insert nucleic acid molecule) such as, but not limited to, a cDNA molecule. Vectors generally comprise parts which mediate vector propagation and manipulation (e.g., one or more origins of replication, genes imparting drug or antibiotic resistance, a multiple cloning site, operatively linked promoter/enhancer elements which enable the expression of a cloned gene, etc.). Vectors may comprise a marker gene that can confer a selectable phenotype, e.g., antibiotic resistance, on a cell. This marker product is used to determine if the gene has been delivered to the cell and once delivered is being expressed. Examples of suitable selectable markers include, but are not limited to, dihydrofolate reductase (DHFR), thymidine kinase, neomycin, neomycin analog G418, hygromycin, blasticidin, and puromycin. When such selectable markers are successfully transferred into a stem cell, the transformed stem cell can survive if placed under selective pressure.

A vector can be a linear molecule, or in circular form, depending on type of vector or type of application. Some circular nucleic acid vectors can be intentionally linearized prior to delivery into a cell. Vector is defined to include any virus, plasmid, cosmid, phage, or binary vector in double or single stranded linear or circular form that may or may not be self-transmissible or mobilizable, and that can transform eukaryotic host cells either by integration into the cellular genome or by existing extrachromosomally (e.g., autonomous replicating plasmid with an origin of replication). One type of vector is an episome, i.e., a nucleic acid capable of extra-chromosomal replication. Another type of vector is one that integrates within the host cell genome. Vectors may be capable of autonomous replication and/or expression of nucleic acids to which they are linked. Protocols for obtaining and using such vectors are known to those in the art.

The term “plasmid” can refer to a DNA molecule with a cell that is physically separated from a chromosomal DNA and can replicate independently.

The term “cosmid” can refer to a plasmid vector that contains a cos sequence.

The term “artificial chromosome” can refer to a nucleic acid sequence of a chromosome that is constructed from a series of smaller nucleic acid sequences. For example, the smaller sequences are constructed into bacterial artificial chromosomes (BAGS) or yeast artificial chromosomes (YACS).

The term “viral vector” can refer to a virus that is competent to infect a mammalian host cell and/or can be used to deliver a construct to a target cells or to an animal systemically.

As used herein, the term “expression vector” can refer to a plasmid origin, a promoter and/or enhancer, one or more transgenes, a transcription terminator, and optionally a selection gene.

The term “genetically-engineered cell” can refer to a cell into which a foreign (i.e., non-naturally occurring) nucleic acid (for example, DNA) has been introduced.

The term “cell” can refer to cytoplasm bound by a membrane that contains DNA within. The cell may be of any organism (e.g., prokaryote, eukaryote, plant, animal) or type (e.g., pluripotent stem cell, differentiated cell, blood cell, skin cell, etc.).

The phrase “cell culture relevant temperatures” can mean any temperature that is known in the art to be appropriate for culturing of cells. “Cell culture relevant temperatures” includes any temperature that is sufficient to maintain the viability of at least one cell during any stage of the cell's life cycle. In embodiments, a cell culture relevant temperature includes any temperature appropriate for generating cell cultures, growing cell cultures, maintaining cell cultures, or a combination thereof. In embodiments, “cell culture relevant temperatures” refers to the temperature at which the cells-of-interest enter a steady state of growth. In embodiments, “cell culture relevant temperatures” include any temperature that is sufficient to maintain the viability of eukaryotic or prokaryotic cells. “Cell culture relevant temperatures” can include any temperature that is sufficient to maintain the viability of mammalian cells. In certain embodiments, “cell culture relevant temperatures” include any temperature that is sufficient to maintain the viability of human cells. “Cell culture relevant temperatures can include any temperature between about 0° C. and about 60° C., inclusive. In embodiments,” cell culture relevant temperatures can include any temperature between about 4° C. and about 42° C., inclusive. “Cell culture relevant temperatures” can include about 20° C., about 21° C., about 22° C. about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C.

As used herein, the expression “operatively linked,” “linked,” “joined,” and similar phrases, when used in reference to nucleic acids, refer to the operational linkage of nucleic acid sequences placed in functional relationships with each other. For instance, if a promoter helps initiate transcription of the coding sequence, the coding sequence can be referred to as operatively linked to (or under control of) the promoter. There may be intervening sequence(s) between the promoter and coding region so long as this functional relationship is maintained.

The term “promoter” refers to a nucleotide sequence, usually upstream (5 prime) of the nucleotide sequence of interest, which directs and/or controls expression of the nucleotide sequence of interest by providing for recognition by RNA polymerase and other factors required for proper transcription. As used herein, the term “promoter” includes (but is not limited to) a promoter that is a short DNA sequence comprised of a TATA-box and other sequences that serve to specify the site of transcription initiation, to which regulatory, or response, elements are added for control of expression. The term “promoter” also refers to a nucleotide sequence that includes a promoter plus regulatory, or response, elements that are capable of controlling the expression of a coding sequence or functional RNA. This type of promoter sequence consists of proximal and more distal upstream elements, the latter elements often referred to as enhancers. The term “enhancer” refers to a DNA sequence that can stimulate promoter activity and can be an innate element of the promoter or a heterologous element inserted to enhance the level or tissue specificity of a promoter. Enhancers are capable of operating in both orientations (normal or flipped) and are capable of functioning even when moved either upstream or downstream of the promoter. Both enhancers and other upstream promoter elements bind sequence-specific DNA-binding proteins that mediate their effects.

A promoter can be derived in its entirety from a native gene, be composed of different elements derived from different promoters found in nature, or even be comprised of synthetic DNA segments. A promoter also can contain DNA sequences that are involved in the binding of protein factors that control the effectiveness of transcription initiation in response to physiological or developmental conditions. Specific promoters used in accordance with the present disclosure can include, for example and without limitation, chicken beta-actin (“CBA”) promoters, cytomegalovirus (“CMV”) promoters, Rous sarcoma virus (“RSV”) promoters, and neuronspecific enolase (“NSE”) promoters.

A cell, tissue, or organism into which has been introduced a foreign nucleic acid, such as a vector, is considered “transformed” or “transfected.” The terms “transforming,” “transfecting,” and the like are used broadly to define a method of inserting or introducing a vector or other nucleic acids into a target cell. This can be accomplished, for example, by transfecting the vector into a target cell. Transfection methods are routine, and a number of transfection methods find use with the invention. These include but are not limited to calcium phosphate precipitation, electroporation, lipid-based methods, cationic polymer transfections, direct nucleic acid injection, biolistic particle injection, and viral transduction using engineered viral carriers (termed transduction, using e.g., engineered herpes simplex virus, adenovirus, adeno-associated virus, vaccinia virus, Sindbis virus), sonoporation, DEAE-mediated transfection, microinjection, retroviral transformation, protoplast fusion, and lipofection. Any of these methods find use with the disclosure.

Transfections can be divided into two categories: stable and transient transfections. Stable transfections result in the vector being permanently introduced into the cell and can be accomplished through the use of selectable marker, e.g., antibiotic resistance. Transient transfections result in the vector being introduced temporarily to the cell. Alternatively, if the vector is a viral vector, it can be transfected into a host cell to produce virus, and the virus can be harvested and used to transduce the vector into the target cell. Transfection and transduction protocols are known in the art.

The embodiments disclosed in the invention may be performed entirely or partially in vivo, in vitro, or a combination thereof.

Before explaining at least one embodiment of the disclosure in detail, it is to be understood that the disclosure is not necessarily limited in its application to the details set forth in the following description or exemplified by the examples. The disclosure is capable of other embodiments or of being practiced or carried out in various ways. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Systems and Methods for Thermostable Expression

In various exemplary embodiments, the disclosed systems and methods enable stable, multigene expression of luciferin/luciferase pathway genes for thermostable protein expression, allowing continuous and/or autonomous light production in the host. Embodiments may be used for small animal or cell-based research and development because certain embodiments provide a means for non-invasively monitoring specific cells in real-time over prolonged time periods.

Certain embodiments include genetically engineered cells and methods of making genetically engineered cells. For example, embodiments express two or more transgenes encoding at least one protein or polypeptide and/or fragments thereof implicated in autonomous bioluminescence within a cell. For example, peptide fragments can comprise functional fragments, such as functional domains of genes involved in the luciferin/luciferase pathway. Embodiments are directed towards compositions and kits comprising the genetically engineered cells, and methods of non-invasively monitoring the genetically engineered cells over prolonged periods and in real-time, such as through the use of bioluminescence.

In embodiments, the method comprises linking multiple luciferase and substrate processing genes using 2A linker regions containing integral protease recognition sites. Although there are a multitude of different strategies for multigene co-expression (e.g., expression as multiple open reading frames with individual promoters, fusion with linking amino acid chains, or IRES elements), 2A elements permit reliable multigene expression in a format amenable to efficient transfection. Counter to the common knowledge in the field—namely, that increasing numbers of 2A-linked open reading frames reduces translational efficiency—it was discovered that sufficiently strong promoters could drive expression of at least six individual open reading frames as a single mRNA (Xu T, Ripp S, Sayler G, Close D. Expression of a humanized viral 2A-mediated lux operon efficiently generates autonomous bioluminescence in human cells. PLoS ONE. 2014; 9(5):e96347). Incorporation of 2A element linkers between open reading frames caused translation of individual proteins from the mRNA.

Unexpectedly, the resulting proteins were highly inefficient at temperatures above 25° C. A variety of hypotheses were explored before discovering that the artifactual C-terminal 2A element amino acids were responsible for this inefficiency. This finding was unexpected not only because physically linked bacterial luciferase proteins have been demonstrated as functional at these temperatures, but also because it contradicted the consensus within the field that 2A linker sequences do not alter the functionality of the up- or downstream protein to which they are appended (Kim J H, Lee S R, Li L H, Park H J, Park J H, Lee K Y, et al. High cleavage efficiency of a 2A peptide derived from porcine teschovirus-1 in human cell lines, zebrafish and mice. PLoS ONE. 2011; 6(4):e18556 and Liu Z, Chen O, Wall J B J, Zheng M, Zhou Y, Wang L, et al. Systematic comparison of 2A peptides for cloning multigenes in a polycistronic vector. Scientific Reports. 2017; 7(1):1-9). Indeed, out of the nearly 1,300 current publications referencing viral 2A linkers, there does not appear to be a single report of the 2A linker sequence interfering with the linked protein's functionality.

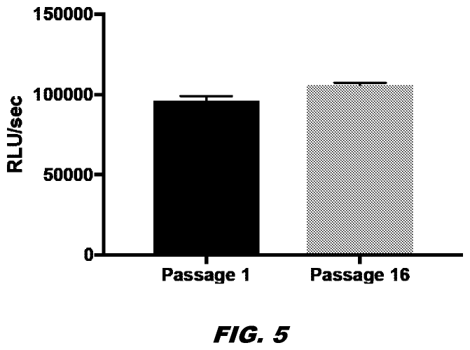

Importantly, incorporation of a protease recognition site between the concluding amino acid residue of the upstream protein and the leading amino acid residue of the 2A linker can be used to remove of the artifactual C-terminal amino acids and the protease recognition site, itself. (Fang J, Yi S, Simmons A, Tu G H, Nguyen M, Harding T C, et al. An antibody delivery system for regulated expression of therapeutic levels of monoclonal antibodies in vivo. Mol Ther. 2007; 15(6):1153-9). As shown in FIGS. 3 and 4, under one embodiment of the presently disclosed system, removal of the artifactual sequences using this process restored functionality to the luciferase/luciferin system at temperatures above 25° C. As shown in FIG. 5, such removal enabled the luciferase/luciferin system to be stably introduced into the cellular genome such that the host cell could continuously or autonomously produce a luminescent signal throughout the cell's lifespan and pass that phenotype to all daughter cells. This discovery significantly improves the utility of cellular assays by providing a means for continuous, non-invasive monitoring of cells using bioluminescence.

In some embodiments the protease recognition sequences are furin recognition sequences. In some embodiments, the protease recognition sequences are: Enterokinase recognition sequences, Factor Xa recognition sequences, Subtilisin BPN′ recognition sequences, TEV recognition sequences, HRV 3C Protease recognition sequences, or similar. The recognition sequence for the employed protease can be chosen from among the full group of amino acid sequences recognized by the desired protease. Each possible amino acid recognition sequence for a given protease may have a different efficiency. One skilled in the art may leverage these efficiency differences to modify the functionality of the system. Similarly, one skilled in the art may select an amino acid sequence such that the residues present contribute in part or in full to function as an alternative functional sequence.

In a basic embodiment, the system can be comprised of repeating genetic structures in the form of an upstream open reading frame, a protease recognition site, a linker region, and a downstream open reading frame, as read in a 5′ to 3′ direction on a sense DNA strand. The downstream open reading frame then serves as the upstream open reading frame of any further repetitions. In this fashion, any number of open reading frames can be linked together such that they produce individual proteins from a single mRNA, with the artifactual amino acids encoded by the protease recognition sequence and the linker region removed by an endogenous protease.

In some embodiments, spacer regions comprise additional nucleotide regions that may be placed between any of the listed elements. These nucleotides can serve to encode additional functionalities, to target the mRNA or protein products to specific locations within the cell or extracellularly, to increase the distance between elements, to impart structures that modify the efficiency of the protease or ribosome at the DNA, RNA, or polypeptide level, to encourage or discourage epigenetic modification, or to encode flexible protein regions that modify the functionality or efficiency of the linker regions. These additional nucleotide regions may function to affect the upstream open reading frame, the downstream open reading frame, distal open reading frames, multiple open reading frames, none of the open reading frames, or any combination thereof.

In some embodiments, the additional nucleotide regions are incorporated into the adjacent open reading frame to function as part of the adjoining protein product. Examples of these include the addition of PEST sequences or other degradation tags to decrease protein half-life. In further embodiments the additional nucleotide regions can comprise binding or purification tags, for example polyhistidine tags or streptavidin or avidin fusion proteins. When placed between the open reading frame and the protease recognition site, the binding properties of these tags are unhindered by the presence of artifactual amino acids resulting from inclusion of the protease recognition sequence and linker region. In further embodiments, the additional nucleotide regions can encode recognitions sequences for DNA-binding proteins, polypeptides, enzymes, DNA, RNA, or non-organic substances.

In some embodiments, the additional nucleotide regions may contain nuclease recognition sequences, meganuclease recognition sequences, or unique nucleotide sequences that can act as barcodes, binding sites for CRISPER/Cas9, transcription activator-like effector nucleases (TALENs), or zinc finger nucleases, transposase recognition sites, viral insertion sites, or similar DNA modification systems. Inclusion of these sequences allows one skilled in the art to easily modify the pathway in question. For example, inserting additional open reading frames, adding or removing stop codons or other regulatory signals, or enabling/disabling alternative splicing of the mRNA.

In some embodiments, upstream of the first open reading frame in the 5′ to 3′ direction on a sense DNA strand can be a promoter, enhancer, operator, or other element capable of initiating or regulating transcription or translation of the downstream open reading frames, or any combination thereof. In some embodiments, downstream of the last open reading frame in the 5′ to 3′ direction on a sense DNA strand can be one or more stop codons, a poly-A sequence, terminator, or other element capable of stopping transcription or translation of the encoded sequence, or any combination thereof.

In some embodiments, the full pathway of interest may be encoded as a single unit for coordinated expression of all pathway open reading frames simultaneously. In other embodiments the pathway of interest may be broken into subsections so that expression of each subsection can be controlled independently. In further embodiments, some or all of the pathway of interest may be expressed using these strategies while relying on traditional exogenous expression of one or more pathway components, or endogenous expression of necessary or equivalent pathway components from the host cell or the environment. One skilled in the art can use these strategies to control relative pathway or exogenous gene expression such that different ratios of transcribed or translated products are produced relative to native or exogenous genes.

In one example of functionality, the bacterial luciferase bioluminescent pathway can be expressed in human cells using this system. The bacterial luciferase bioluminescent pathway presents a suitable example because it comprises multiple exogenous genes and does not function efficiently at the mammalian growth temperature optimum of 37° C. if stably expressed using traditional approaches. In fact, this approach is the only known method for enabling functional, stable expression of the bacterial luciferase bioluminescent pathway in human cells.

In this example, the bacterial luciferase pathway genes or lux cassette (i.e., luxC, luxD, luxA, luxB, and luxE), and a supporting oxidoreductase gene, frp, can be codon optimized for expression in HEK293 cells. The stop codons can be removed from the luxC, luxD, luxA, luxB, and luxE genes. A Furin protease recognition sequence (R-K-R-R) followed by a T2a 2A linker can be placed between the luxC and luxD genes. A Furin protease recognition sequence (R-K-R-R) followed by a E2a 2A linker can be placed between the luxD and luxA genes. A Furin protease recognition sequence (R-K-R-R) followed by a P2a 2A linker was placed between the luxA and luxB genes. A Furin protease recognition sequence (R-K-R-R) followed by a Pa2a 2A linker (comprising a P2a 2A linker amino acid sequence encoded by an alternative DNA sequence) can be placed between the luxB and luxE genes. A Furin protease recognition sequence (R-K-R-R) followed by a FMDV 2A linker can be placed between the luxE and frp genes. This full sequence can be placed under the control of a CMV IE enhancer and CMV IE promoter and transfected into HEK293 cells. Autonomously bioluminescent isolates were selected based on light output and resistance to G418 as encoded by a selection marker on the delivery vector.

Stably selected cells developed using this method are capable of autonomously producing a bioluminescent signal when cultured at 37° C. (see FIG. 4). This is a significantly different result than can be achieved using alternative strategies, such as expressing the bacterial luciferase genes from individual promoters, using IRES elements to express multiple bacterial luciferase genes, or linking bacterial luciferase genes with 2A linkers without protease recognition sequences; all of which fail to either stably express the bacterial luciferase bioluminescent pathway, or stably express the pathway but prevent efficient generation of a bioluminescent signal at 37° C.

As an alternative example, this strategy can be used to stably express the fungal luciferase bioluminescent pathway in eukaryotic cells. Like the bacterial luciferase bioluminescent pathway, the fungal luciferase bioluminescent pathway comprises multiple exogenous genes. However, in this example, the genes are sourced from multiple different organisms. In this example, a Rhodobacter capsulatus tyrosine ammonia lyase and two Escherichia coli 4-hydroxyphenylacetate 3-monooxygenase components are linked with the fungal genes npgA, hisps, h3h, and luz using intervening protease recognition sequences and 2A linkers. As before, this approach allows the individual open reading frames to be transcribed as a single mRNA, translated as individual proteins, and then processed by endogenous proteases such that the artifactual amino acids from the protease recognition and 2A linker sequences are removed.

This approach could also be applied to bioluminescent systems with more complex expression pathways, such as the luciferase pathways from fireflies, sea pansies, copepods, or dinoflagellates. Due to the complexity of these pathways, multiple strategies can be used. As one example, the full complement of genes required for luciferase, luciferin, and supporting analyte processing could be encoded as a single operon with intervening protease recognition sequences and 2A linkers. In another example, only those proteins without homologs in the host cell could be encoded as a single operon with intervening protease recognition sequences and 2A linkers, while the functions of the non-encoded open reading frames are performed by native homologs from the host cell. In another example, portions of the pathway are expressed individually, while other portions are encoded as a single operon with intervening protease recognition sequences and 2A linkers. In a further example, any combination of these strategies may be employed to achieve pathway functionality.

This approach is not limited to luciferase/luciferin pathway expression and can be used for thermostable expression of any multigene system. In a basic example, the approach can be used to express an upstream gene of interest with a downstream fluorescent reporter gene, such as GFP, YFP, RFP, mOrange, mCherry, dsRed, or similar. This configuration allows thermostable expression of the upstream gene of interest in its native form and expression of the downstream reporter protein to positively identify cells actively transcribing and translating the gene of interest and/or quantify transcriptional/translational levels of the gene of interest by measuring the fluorescent output of the downstream reporter. In a more complicated extension of this example, multiple genes of interest can be linked upstream of a reporter gene to enable similar capabilities with a more complex pathway. In some embodiments of this example, multiple fluorescent reporter genes can be interspersed among the genes of interest to enable estimation of the transcriptional/translational levels of one or more genes along the pathway.

Without being bound by theory, in an embodiment of this example, the approach can be used to restore correct protein targeting by obviating the disruption of signal proteins resulting from association with 2A linkers. For example, when a fluorescent reporter gene, dsRed, with a C-terminal peroxisome targeting sequence is upstream of a second fluorescent reporter gene, GFP, without a targeting sequence using a 2A linker, the dsRed protein can fail to localize to the peroxisome and is expressed cytosolically similarly to the untagged GFP protein because the presence of the artifactual amino acids from the 2A linker modified the C-terminus of the protein such that the peroxisome targeting sequence can no longer be recognized by its receptor protein. However, without being bound by theory, adding an intervening protease recognition sequence upstream of the 2A linker will permit protease cleavage-mediated removal of the artifactual amino acids and will restore the correct positioning of the peroxisome targeting sequence. As a result, functionality can be restored and the dsRed protein can be correctly trafficked to the peroxisome.

In other embodiments of this example, the reporter gene could be substituted for an antibiotic resistance gene. Placing the antibiotic resistance gene downstream of the gene(s) of interest with an intervening protease recognition sequence and 2A linker allows thermostable expression of the gene(s) of interest in their native forms and expression of the antibiotic resistance protein allows one to positively identify cells actively transcribing and translating the gene(s) of interest and/or stably selection and propagation of clonal lineages of those cells. In other embodiments, the gene(s) encoding antibiotic resistance may be expressed separately from the genes of interest.

Without being bound by theory, the system could be used to simultaneously express thermostable versions of the four Yamanaka reprogramming factor genes: Oct-4, Sox2, Klf4, and c-Myc as a single operon with intervening protease recognition sequences and 2A linkers. This approach is advantageous relative to alternative approaches in that all four of the genes could be placed under the control of an inducible promoter to enable precise control over expressional timing. The ability to stably express thermostable versions of these proteins with a single point of control is advantageous for regenerative medicine, developmental biology, cellular biology, and basic research, and related fields of study.

The system can also have clinical or therapeutic applications. In clinical or therapeutic applications, it is paramount that proteins be expressed in their native form or without unintended modifications to their desired form. Furthermore, deployment of gene therapies within human subjects requires that the employed protein products remain thermostable and are expressed in a controlled fashion. The use of this system of open reading frames interspersed with intervening protease recognition sequences and 2A linkers allows these criteria to be met. In a generalized example of this application, a patient deficient in the expression of multiple genes could be treated with DNA or RNA encoding the deficient gene products. Upon translation, the presence of intervening protease recognition sequences and 2A linkers among the open reading frames would result in thermostable versions of the target proteins without artifactual amino acids that could modify their functionality or longevity. Furthermore, as described in previous examples, one skilled in the art could leverage the relative orientation of these ORFs, the presence or absence of additional nucleotide regions, combinations of these factors, or other similar control strategies to fine tune the transcription and/or translation of the nucleotides to improve therapeutic outcomes.

Open Reading Frames and Transgenes

In some embodiments, the ORFs or transgenes of the present disclosure may encode a polypeptide comprising a multigene pathway. In embodiments, the multigene pathway comprises luciferin/luciferase pathway genes and/or fragments thereof. In embodiments, the polypeptide comprises luxC, luxD, luxA, luxB, luxE, luxF, luxG, luxH, luxI, luxR, luxY, frp, luz, H3H, or HipS, CPH, npgA, TAL, hpaB, hpaC, fragments of any of the foregoing, or combinations thereof. For example, the polypeptide may comprise SEQ ID NO: 1, SEQ ID NO: 2, SEQ ID NO: 3, SEQ ID NO: 4, SEQ ID NO: 5, SEQ ID NO: 6, or SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14, SEQ ID NO: 15, SEQ ID NO: 65, SEQ ID NO: 66, SEQ ID NO: 67, SEQ ID NO: 68, SEQ ID NO: 69, fragments of any of the foregoing, or a combination thereof.

In certain embodiments, the polynucleotide comprises at least 80% identity to any one or more of the following nucleic acid sequences: SEQ ID NO: 33, SEQ ID NO: 34, SEQ ID NO: 35, SEQ ID NO: 36, or SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 70, SEQ ID NO: 71, SEQ ID NO: 72, SEQ ID NO:73, SEQ ID NO: 74, fragments of any of the foregoing, or a combination thereof. In embodiments, the polynucleotide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one or more of the following nucleic acid sequences: SEQ ID NO: 33, SEQ ID NO: 34, SEQ ID NO: 35, SEQ ID NO: 36, or SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and SEQ ID NO: 47.

In certain embodiments, the transgene can comprise a fluorescent reporter gene or fragments thereof. In embodiments, the fluorescent reporter gene comprises GFP, YFP, RFP, dsRed, mOrange, mCherry, fragments of any of the foregoing, or combinations thereof. By way of example, the polypeptide can comprise SEQ ID NO: 16, EQ ID 17, SEQ ID NO: 18, SEQ ID NO: 19, SEQ ID NO: 20, SEQ ID NO: 21, fragments of any of the foregoing, or a combination thereof.

In certain embodiments, the polynucleotide comprises at least 80% identity to any one or more of the following nucleic acid sequences: SEQ ID NO: 48, SEQ ID NO: 49, SEQ ID NO: 50, SEQ ID NO: 51, SEQ ID NO: 52, SEQ ID NO: 53, fragments of any of the foregoing, or a combination thereof. In embodiments, the polynucleotide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one or more of the following nucleic acid sequences: SEQ ID NO: 48, SEQ ID NO: 49, SEQ ID NO: 50, SEQ ID NO: 51, SEQ ID NO: 52, and SEQ ID NO: 53.

The transgene can comprise a Yamanaka reprogramming factor gene. In certain embodiments, the Yamanaka reprogramming factor gene comprises Oct-4, Sox2, Klf4, c-Myc, fragments of any of the foregoing, or combinations thereof. By way of example, the polypeptide can comprise SEQ ID NO: 22, SEQ ID NO: 24, SEQ ID NO: 25, fragments of any of the foregoing, or a combination thereof.

In certain embodiments, the polynucleotide comprises at least 80% identity to any one or more of the following nucleic acid sequences: SEQ ID NO: 54, SEQ ID NO: 55, SEQ ID NO: 56, SEQ ID NO: 57, fragments of any of the foregoing, or a combination thereof. In embodiments, the polynucleotide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one or more of the following nucleic acid sequences: SEQ ID NO: 54, SEQ ID NO: 55, SEQ ID NO: 56, SEQ ID NO: 57.

In further embodiments, the transgene or ORF may include a polypeptide or nucleic acid sequence that is not naturally found in the cell (i.e., a heterologous nucleic acid sequence). A transgene or ORF can further include non-coding sequences such as ribozymes or guide RNAs (gRNAs) for use in nucleic acid editing assays such as the CRISPR/Cas systems.

In embodiments, the transgene can comprise a synthetic polynucleotide, which can refer to a polynucleotide sequence that does not exist in nature but instead is made by the hand of man, either chemically, or biologically (i.e., in vitro modified). For example, the synthetic polynucleotide can be made using cloning and vector propagation techniques.

Vectors can be used to transport the insert nucleic acid molecule into a suitable host cell. A vector can contain the elements necessary to permit transcribing the insert nucleic acid molecule, and, optionally, translating the transcript into a polypeptide. The insert nucleic acid molecule can be derived from the host cell or may be derived from a different cell or organism. Once in the host cell, the vector can replicate independently of, or coincidental with, the host chromosomal DNA, and several copies of the vector and its inserted nucleic acid molecule may be generated (Sambrook et al., Molecular Cloning: A Laboratory Manual, Cold SpringHarbor Laboratory, Cold Spring Harbor, 1989).

In further embodiments, the vector can include both non-viral and viral vectors. Non-viral vectors include but are not limited to cationic lipids, liposomes, nanoparticles, PEG, and PEI. Viral vectors are derived from viruses and include but are not limited to retrovirus, lentivirus, adeno-associated virus, adenovirus, herpesvirus, and hepatitis virus. Viral vectors can be replication-deficient as they have lost the ability to propagate in a given cell since viral genes essential for replication have been eliminated from the viral vector. However, some viral vectors can also be adapted to replicate specifically in a given cell, such as, for example, a cancer cell.

In embodiments, vectors can be derived from adeno-associated virus, adenovirus, retroviruses and Antiviruses. Alternatively, gene delivery systems can be used to combine viral and non-viral components, such as nanoparticles or virosomes (Yamada, Tadanori, et al. “Nanoparticles for the delivery of genes and drugs to human hepatocytes.” Nature biotechnology 21.8 (2003): 885-890). Retroviruses and Antiviruses are RNA viruses that have the ability to insert their genes into host cell chromosomes after infection. Retroviral and lentiviral vectors have been developed that lack the genes encoding viral proteins, but retain the ability to infect cells and insert their genes into the chromosomes of the target cell (Miller, Daniel G., Mohammed A. Adam, and A. Dusty Miller. “Gene transfer by retrovirus vectors occurs only in cells that are actively replicating at the time of infection.” Molecular and cellular biology 10.8 (1990): 4239-4242; Naldini, Luigi, et al. “In vivo gene delivery and stable transduction of nondividing cells by a lentiviral vector.” Science 272.5259 (1996): 263., VandenDriessche, Thierry, et al. “Long-term expression of human coagulation factor VIII and correction of hemophilia A after in vivo retroviral gene transfer in factor VIII-deficient mice.” Proceedings of the National Academy of Sciences 96.18 (1999): 10379-10384.). The difference between a lentiviral and a classical Moloney-murine leukemia-virus (MLV) based retroviral vector is that lentiviral vectors can transduce both dividing and non-dividing cells whereas MLV-based retroviral vectors can only transduce dividing cells.

The genetically engineered cell of the claimed invention can express transgenes as described herein from vectors, non-limiting examples of which comprise viral vectors, plasmids, such as bacterial plasmids, cosmids, and artificial chromosomes. One example of a viral vector is the first generation E1/E3 deleted nonreplicating Ad5 vector, but other forms of viral delivery systems are known and could be used. One of the disadvantages of the non-replicating adenovirus is the lack of persistence in vivo and one embodiment could be the use of a conditionally replicating oncolytic adenovirus. Additional examples of viral delivery systems comprise viruses that would result in more permanent expression such as lentivirus or adeno-associated virus (AAV). The advantage to these two viral systems is that they can be manipulated to alter their tropism for different cell types making them a more flexible platform.

There are several types of viral vectors that can be used to deliver nucleic acids into the genetic makeup of cells, non-limiting examples of which include retrovirus, lentivirus, adenovirus, adeno-associated virus and herpes simplex virus. For example, the vector can be a lentiviral vector, such as pReceiver.

Such vectors, also known as expression vectors or DNA expression constructs, can be modified to include and/or be operatively linked to regulatory elements to carry out the embodiments of this invention. Additionally, such vectors can contain multipurpose cloning regions that have numerous restriction enzyme sites.

Embodiments can contain markers for selection of cells that are positively transfected with the vector. Non-limiting examples of such selection markers include antibiotic resistant genes, such as those that result in resistance to neomyocin, puromycin, G418, or ampicillin, or fluorescent markers, such as mCherry or EGFP, or a combination of selections markers.

Linkers

The described system provides advantages over previous systems and alternative approaches. Using linker regions resulting in independent proteins, rather than physically linked proteins or functional units, enables the resulting protein products to take advantage of intracellular environmental dynamics for access to intracellular materials and prevents interactional inhibition due to steric limitations. Furthermore, it allows multiple functional units to be delivered to a cell simultaneously, enables ratio-based introduction of DNA sequences for copy number control, and provides a facile method for coordinated regulation of subsets of the expressed cohort.

Relative to alternative approaches, using 2A linkers reduces the length of DNA that must be introduced and incorporated into the cellular genome to achieve pathway expression, which improves the efficiency of the transfection and selection processes. The variety of different 2A linker sequences available ensures that repetitive DNA sequence utilization, which can result in unintended natural modification within the host and increase the difficulty of genetic manipulation at the bench, can be avoided. For one skilled in the art, the differential efficiencies of available 2A linkers can also be used to modify transcriptional expression ratios of the linked open reading frames through rational design of the pathway expression order.

In some embodiments the linker regions used are 2A linker regions. The 2A linker regions can include, but are not limited to, T2a, E2a, F2a, P2a, FMDV2a, or similar. The linker regions can comprise SEQ ID NO: 28, SEQ ID NO: 29, SEQ ID NO: 30, SEQ ID NO: 31, SEQ ID NO: 32, fragments of any of the foregoing, or combinations thereof.

In certain embodiments, the polynucleotide comprises at least 80% identity to any one or more of the following nucleic acid sequences: SEQ ID NO: 60, SEQ ID NO: 61, SEQ ID NO: 62, SEQ ID NO: 63, SEQ ID NO: 64, fragments of any of the foregoing, or a combination thereof. In embodiments, the polynucleotide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one or more of the following nucleic acid sequences: SEQ ID NO: 60, SEQ ID NO: 61, SEQ ID NO: 62, SEQ ID NO: 63, and SEQ ID NO: 64.

For some linker regions, it may be necessary to introduce amino acid substitutions to the canonical linker sequence so that undesired protease recognitions motifs are avoided or secondary structures are not formed, to abolish potential binding interactions, or prevent similar unwanted functionality. For some linker regions, it may be desirable to leverage a naturally occurring protease recognition site within the linker either in place of, or in addition to, a separately incorporated protease recognition site. For some linker regions, it may not be necessary to impart any modifications. One skilled in the art can use the presence and/or absence of individual or multiple modifications to change the location and/or efficiency of the protease recognition sequence to fine tune its functionality within the system.

Protease Recognition Sequences

The use of protease recognition sequences provides a simplistic method for removing artifactual amino acid residues from the expressed proteins and thereby increasing the likelihood of wild type functionality. The advantages of protease recognition sequences, such as Furin recognition sequences, parallel those of the 2A linker regions. In certain embodiments, the protease recognition sequence encodes for SEQ ID NO: 26.

In certain embodiments, the protease recognition sequence polynucleotide comprises at least 80% identity to SEQ ID NO: 58 or any fragment thereof. In embodiments, the polynucleotide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 58:

Protease recognition sequences can be encoded using a short DNA sequence. Multiple DNA and amino acid identities are available to enable codon optimization while avoiding repetitive DNA sequences, and there is a large body of research available to inform sequence design relative to the surrounding amino acid residues in order to modulate efficiency. Also similar to the use of 2A linkers, they are non-coding sequences and function entirely using host machinery. This limits the number of exogenous genes that must be introduced to enable system functionality and therefore limits the impact of exogenous expression on the host.

Genetically Engineered Cells

Embodiments of the present invention are directed towards a genetically engineered cell line configured to permit thermostable expression of a multigene system. Certain embodiments comprise a plurality of cells transformed with at least one polynucleotide encoding a protein, polypeptide, or fragment thereof involved in bioluminescence. In some embodiments, the protein, polypeptide, or fragment thereof is involved in the luciferin/luciferase pathway.

In order to generate the genetically engineered cell as described under embodiments herein, each of the following can be introduced into at least one cell: at least two polynucleotides encoding proteins, polypeptides, or fragments thereof that are involved in a multigene system, at least one 2A linker, and at least one protease recognition site.

The polynucleotide, which can comprise DNA, RNA, or a fragment thereof, can be introduced into a cell of any cell type. A cell can be either a prokaryotic or eukaryotic cell. In some embodiments, the cell can be isolated from a tissue from a human subject. Non-limiting examples of such tissues comprise skin, kidney, adipose tissue, bone marrow, blood, human brain cells, pericytes, macrophages, or retinal pigment epithelial cells. In other embodiments, the cell may be of any of the following cell types: skin fibroblasts, adipose tissue stem cells, primary retinal pigment epithelial cells, human embryonic cells, human adult stem cells, transdifferentiated neuronal cells, pericytes, and macrophages.

Further, the plurality of cells can be a stem cell, such as a pluripotent stem cell or a totipotent stem cell. The stem cell may be any type of stem cell, for example, an adult stem cell (e.g., a tissue-specific stem cell), an embryonic (or pluripotent) stem cell, and an induced pluripotent stem cell (iPSC). The term “stem cell” also includes any progeny, and it is understood that all progeny may not be identical to the parental cell since there may be mutations that occur during replication. Exemplary but non-limiting established lines of human embryonic stem (ES) cells include lines which are listed in the NIH Human Embryonic Stem Cell Registry (http://stemcells.nih.gov/research/registry), and sub-lines thereof. Other exemplary established hES cell lines include those deposited at the UK Stem Cell Bank (http://www.ukstemcellbank.org.uk/), and sub-lines thereof.

Stem cells may include cells, such as progenitor cells, further capable of self-renewal, which can under appropriate conditions proliferate without differentiation. Stem cells can also be cells capable of substantial unlimited self-renewal, wherein at least a portion of the stem cell's progeny substantially retains the unspecialized or relatively less specialized phenotype, the differentiation potential, and the proliferation capacity of the mother stem cell. Stem cells can also be cells which display limited self-renewal, wherein the capacity of the stem cell's progeny for further proliferation and/or differentiation is demonstrably reduced compared to the mother cell.

Pluripotent stem cells are capable of giving rise to cell types originating from all three germ layers of an organism (i.e., mesoderm, endoderm, and ectoderm), and potentially capable of giving rise to any and all cell types of an organism, although not able to grow into the whole organism.

A progenitor or stem cell can refer to a cell that can “give rise” to another, relatively more specialized cell when, for example, the progenitor or stem cell differentiates to become said other cell without previously undergoing cell division, or if said other cell is produced after one or more rounds of cell division and/or differentiation of the progenitor or stem cell. A “mammalian pluripotent stem cell” or “mPS” cell can refer to a pluripotent stem cell of mammalian origin. Animals of “mammalian origin” can refer to any animal classified as such, non-limiting examples of which include humans, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates (e.g., monkeys and apes).

In other embodiments, the plurality of cells can be populations of cells, and subpopulations thereof, such as those distinguished and isolated from a sample population.

Without wishing to be bound by theory, the plurality of cells can comprise any cells that have characteristics of mammalian cells (i.e. mouse or human cells) or pluripotent cells (i.e., embryonic stem cells or embryonic germ cells).

Methods of Generating the Genetically Engineered Cells

The invention also provides for methods of generating genetically engineered cells as described herein. For example, an embodiment comprises the step of obtaining a plurality of cells and introducing into the cells each of the following: at least two multigene system polynucleotides, each encoding at least one polypeptide involved in a multigene system; at least one linker polynucleotide encoding a 2A linker; and at least one protease polynucleotide encoding a protease recognition site. Embodiments further comprise placing the at least one linker polynucleotide between the at least two multigene system polynucleotides and placing the protease polypeptide between one of the at least two multigene system polynucleotides and the linker polynucleotide.

Embodiments can further comprise detecting the presence of the expression vector or the polypeptide within the plurality of cells, for example, by antibiotic resistance screens, immunohistochemistry (such as Western blot analysis), or FACS. Also, the biological functions of the polypeptides can be confirmed, such as by detecting bioluminescence.

In embodiments, the polynucleotide can be introduced into the cells by transduction, such as transfer by bacteriophages or viruses; transformation, such as uptake of naked DNA from outside of the cell; microinjection; or any other means of introducing the polynucleotide into the cells.

As discussed herein, functionality of certain multigene pathways can be impaired at cell culture relevant temperatures when the multigene pathway is introduced to the host cell using a 2A linker-based approach. As further discussed, this thermoinstability can be remedied by removal of the artifactual C-terminal residues of the 2A linker sequence. In embodiments, incorporation of a protease recognition site between the concluding amino acid residue of the upstream protein and the leading amino acid residue of the 2A linker allows for removal of the artifactual C-terminal amino acids and the protease recognition site itself and permits thermostable functionality of the transfected gene pathway.

In one embodiment, a pCMVₗᵤₓ vector, which contains the luxC, luxD, luxA, luxB, luxE, and frp genes required for autonomous bioluminescent production linked by viral 2A element spacers can be used as the basis for developing an improved vector with self-cleaving, 2A-linked sequences. In this embodiment, a luxC-linker-luxD-linker fragment, a luxA-linker-luxB fragment, and a linker-luxE-linker-frp fragment can be synthesized such that a Furin recognition sequence is incorporated in frame directly upstream of each linker region. The individual segments can then be linked together and assembled into the pCMVₗᵤₓ backbone in place of the original 2A-linked luciferin/luciferase pathway cassette using a HiFi DNA Assembly reaction.

HEK-293 cells can be cultured in a humidified incubator at 37° C. with 5% CO 2 and grown in Dulbecco's Modified Eagle Medium (DMEM) supplemented with 10% Fetal Bovine Serum (FBS), 1×Penn/Strep (ThermoFisher), and 1×GlutaMAX (ThermoFisher). HEKs can be plated at 10,000 cells/well in a 96 well plate 24 hours prior to transfection. Transfection mixes can be prepared by combining 100 ng of DNA with Viafect Transfection Reagent. The transfection mixes can be incubated at room temperature for 10 minutes then added dropwise to HEKs. BMG LABTECH's CLARIOstar can be used to analyze transfected HEKs at 24 and 48 hours post transfection using 1 second integration at 25 or 37° C.

Bioluminescence

Certain embodiments of the presently disclosed system provide for thermostable expression of multigene pathways involved in bioluminescence in a host cell. One embodiment permits thermostable expression of bacterial luciferin/luciferase pathways. Functional bacterial luciferase requires expression of luxC, luxD, luxA, luxB, luxE, and frp. Cells (such as stem cells) including nucleic acids encoding each of luxA, luxB, luxC, luxD, luxE, and flavin reductase autonomously produce a bioluminescent signal via luxA, luxB, luxC, luxD, luxE, and flavin reductase working synergistically with endogenous myristic acid, endogenous flavin mononucleotide, and molecular oxygen to generate the bioluminescent signal.

Specifically, luxC, luxD, and luxE form a tetra-quatramer that processes natural cellular metabolites into the aldehyde luciferin. LuxA and luxB form a dimer that functions as the luciferase. Frp recycles the supporting metabolite, FMNH₂, after it is oxidized to FMN in the bioluminescent reaction (Meighen E A. Molecular biology of bacterial bioluminescence. Microbiological Reviews. 1991; 55(1):123-42). For expression in eukaryotic organsisms, the tetra-quatramer formed by the LuxC, LuxD, and LuxE proteins converts myristol-ACP intented for membrane biogenesis into myristal aldehyde to act as a substrate for the bioluminescent reaction (Close D M, Ripp S, Sayler G S. Reporter proteins in whole-cell optical bioreporter detection systems, biosensor integrations, and biosensing applications. Sensors. 2009; 9(11):9147-74). The heterodimer formed by LuxA and LuxB is capable of functioning agnostically of the host, so long as it is provided with the aldehyde, oxygen, and FMNH₂, the latter of which are naturally available within human cells. Frp then functions to recycle oxidized FMN into FMNH₂, similarly to its role in prokaryotic organisms (Lin L Y C, Sulea T, Szittner R, Vassilyev V, Purisima E O, Meighen E A. Modeling of the bacterial luciferase flavin mononucleotide complex combining flexible docking with structure activity data. Protein Sci. 2001; 10(8):1563-71). Therefore, the coexpression of luxA, luxB, luxC, luxD, luxE, and flavin reductase allows the cell to generate a bioluminescent signal in a fully autonomous fashion (that is, without the addition of an exogenous reagent). The overall reaction can be summarized as: FMNH₂+RCHO+O₂→FM N-FH₂O+RCOOH+hv490 nm.

In the present systems, nucleic acid cassettes can be designed to match this native gene order as generally discussed above. However, such an order is not required to maintain functionality of the presently disclosed system. For example, the order of the genes can be modified to place the luxC gene, which is traditionally the gene closest to the promoter, at the distal end of the cassette such that is arranged luxD, luxA, luxB, luxE, frp, luxC. Another embodiment permits thermostable expression of fungal luciferin/luciferase pathways. The luciferase in this system can be encoded by the luz gene. In addition, two luciferin synthesis genes: hisps and h3h, work together to as a polyketide synthase and a 3-hydroxybenzoate 6-monooxygenase to supply the required luciferin, 3-hydroxyhispidin. For autonomous function in cells that do not naturally produce the supporting analyte, caffeic acid, this pathway can also be encoded with genes for tyrosine ammonia lyase, two 4-hydroxyphenylacetate 3-monooxygenase components and the 4′-phosphopantetheinyl transferase gene npgA (Kotlobay A A, Sarkisyan K S, Mokrushina Y A, Marcet-Houben M, Serebrovskaya E O, Markina N M, et al. Genetically encodable bioluminescent system from fungi. Proceedings of the National Academy of Sciences of the United States of America. 2018; 115(50):12728-32). The luciferase and/or luciferin processing proteins can be multimers formed by the products of multiple genes.

### Methods of Use

In one embodiment, the invention provides a method of non-invasive cellular monitoring. In embodiments, the methods provide for continuous, non-invasive monitoring of cells in real-time. This method of use can provide for cellular monitoring over long periods of time. In embodiments, the method provides for identification of cells involved in active transcription of a gene of interest, translation of a gene of interest, or a combination thereof.

In some embodiments, the method of non-invasive cellular monitoring may also include providing at least one cell producing bioluminescence, wherein the cell has been transfected with any of the nucleic acid constructs disclosed herein; and monitoring the bioluminescence of the cell. The bioluminescence may be detectable at multiple time points and in real-time. In some embodiments, the bioluminescence is detectable in the absence of an exogenous luminescent stimulator, i.e., the signal is produced “autonomously.” The exogenous luminescent stimulator may be a fluorescent stimulation signal. The exogenous luminescent stimulator may be a chemical luminescent activator. In some embodiments, the chemical luminescent activator may comprise a luciferin or luciferin analog. For example, in some embodiments, the chemical may comprise, at least, an aldehyde functional group. In other embodiments, the chemical luminescent activator may comprise, for example, D-luciferin (2-(4-hydroxybenzothiazol-2-yl)-2-thiazoline acid), 3-hydroxy-hispidin, coelenterazine, or any other luciferin substrate.

Given the ability of the autonomously bioluminescent cell to produce bioluminescence without the need for an investigator to add an exogenous substrate, the cell has applications in, for example, real-time, non-invasive, continuous, and substrate-free tracking, identifying, and/or measuring the cells' viability, migration, and/or fate. In some embodiments, the present disclosure provides methods of real-time monitoring of cell population size of a population of at least one cell producing bioluminescence, wherein the cell has been transfected with any of the nucleic acid constructs disclosed herein. In further embodiments, the present disclosure provides methods of real-time monitoring of cell viability of at least one cell producing bioluminescence, wherein the cell has been transfected with any of the nucleic acid constructs disclosed herein. The methods may comprise detecting, measuring, and/or quantifying the bioluminescence emitted from the at least one cell by any device suitable for detecting, measuring, and/or quantifying the bioluminescence. The detection, measurement, and/or quantification may occur at one or more time points.

In further embodiments, the presently disclosed methods permit quantification of transcription levels of a gene of interest, translation levels of a gene of interest, or a combination thereof. By way of example, the method can comprise thermostably expressing a gene of interest with a downstream fluorescent reporter gene and identifying the fluorescent reporter gene, wherein fluorescence indicates which cells are actively involved with transcription of the gene of interest, translation of the gene of interest, or a combination thereof. Certain embodiments comprise quantifying the degree of transcription of the gene of interest, the degree of translation of the gene of interest, or a combination thereof, wherein an increased level of fluorescence indicates an increased level of transcription of the gene of interest, translation of the gene of interest, or a combination thereof. Multiple genes of interest can be linked upstream of a reporter gene to enable similar capabilities with complex pathways. In some embodiments, multiple fluorescent reporter genes can be interspersed among the genes of interest to enable estimation of the transcriptional/translational levels of one or more genes along the pathway.

Another method of use comprises confirming correct localization of a gene of interest. For example, the method can comprise forming a nucleic acid cassette by using a 2A linker to place a fluorescent reporter gene comprising with a C-terminal peroxisome targeting sequence upstream of a second fluorescent reporter gene, wherein the second fluorescent report gene lacks a peroxisome targeting sequence. Embodiments further comprise adding an intervening protease recognition sequence upstream of the 2A linker, introducing the nucleic acid cassette into a host cell, and permitting protease cleavage to remove the 2A C-terminal artifactual amino acids. The method can further comprise quantifying the amount of the first fluorescent reporter gene present within the peroxisome of the host cell to confirm the relative amount of trafficking to the peroxisome.

An alternate method of use comprises placing an antibiotic resistance gene downstream of one or more genes of interest with an intervening protease recognition sequence and 2A linker to and introducing the nucleic acid cassette into a host cell to permit thermostable expression of the one or more genes of interest in a host cell their native forms. The method can further comprise positively identifying cells actively transcribing the one or more genes of interest, translating the one or more genes of interest, or a combination thereof, wherein expression of the antibiotic resistance protein indicates which cells are actively transcribing and translating the one or more genes of interest. The method can further include stably selecting and propagating clonal lineages of those cells that actively transcribe and translate the one or more genes of interest. In other embodiments, the method comprises expressing the gene encoding antibiotic resistance separately from the one or more genes of interest.

Another method of use comprises treating a patient who has a deficiency in expression of one or more genes. In such embodiments, the treatment can comprise providing the patient with DNA or RNA ORFs encoding the deficient gene products, wherein the DNA or RNA ORFs are interspersed with intervening protease recognition sequences and 2A linkers as described herein. Embodiments further include permitting transcription and translation of the one or more genes into target proteins, wherein the presence of intervening protease recognition sequences and 2A linkers among the open reading frames results in thermostable versions of the target proteins that lack artifactual amino acids, which could otherwise modify target protein's functionality or longevity. Furthermore, as described in previous examples, one skilled in the art could leverage the relative orientation of these ORFs, the presence or absence of additional nucleotide regions, combinations of these factors, or other similar control strategies to fine tune the transcription and/or translation of the nucleotides to improve therapeutic outcomes.

Kits Comprising Nucleic Acid Cassettes and/or Genetically Modified Cells

The invention also provides for a kit for using any of the various nucleic acid cassettes or genetically modified cells lines described herein.

The kit can be used to carry out any of the various methods as described herein.

The genetically engineered cells can be packaged in the kit by any suitable means for transporting and storing cells. For example, the cells can be provided in frozen form, such as cryopreserved; dried form, such as lyophilized; or in liquid form, such as in a buffer. Cryopreserved cells, for example, can be viable after thawing.

The kits may include instructions. The instructions may include one or more of: a description of the genetically engineered cells; methods for thawing or preparing cells; precautions; warnings; animal pharmacology; clinical studies; and/or references. The instructions can be printed directly on the container (when present), or as a label applied to the container, or as a separate sheet, pamphlet, card, or folder supplied in or with the container. Generally, a kit as described herein also includes packaging. In some embodiments, the kit includes a sterile container which contains a genetically engineered cells; such containers can be boxes, ampules, bottles, vials, 10 tubes, bags, pouches, blister-packs, or other suitable container forms known in the art. Such containers can be made of plastic, glass, laminated paper, metal foil, or other materials suitable for holding cells or medicaments.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Abstract: A system for stable, thermostable expression of luciferase/luciferin pathway genes and proteins in eukaryotic cells is disclosed. The system enables multigene pathways encoding some or all of a series of luciferase, luciferin, and supporting analyte proteins to be expressed and maintain functionality at cell culture relevant temperatures. The disclosed system provides a means for generating eukaryotic cells capable of continuous or autonomous light production and control of expression in response to physiological changes.

There is a clear need for low cost, high-throughput, non-invasive cellular monitoring methods in biological fields such as drug development, toxicology, environmental monitoring, and basic research. Bioluminescence, the production of light from a living cell, would be an ideal detection modality for these applications, but has not been employed because it is limited to only single time points, requires expensive externally applied reagents to function across a limited time span, or cannot be exogenously expressed at temperatures relevant for most applications. Adaption of the system to stably function continuously or autonomously solves these problems.

For a cell to autonomously produce a luminescent signal, it must express genes for both the luciferase enzyme and the proteins required for substrate production, trafficking, and regeneration. These pathways may require co-expression of more than one gene. Modulation, or lack thereof, of the luminescent phenotype may require dependent or independent expressional control of individual luciferase or substrate processing genes, groups of luciferase or substrate processing genes, or the full pathway of luciferase and substrate processing genes. Co-expression may require genes to be linked to enable multiple proteins to be obtained from a single mRNA sequence.

Luminescent systems with known luciferin/luciferase pathways, such as bacterial luciferase or fungal luciferase, require expression of multiple genes to enable autonomous bioluminescent production. Efficient introduction of these multiple genes into naturally non-luminescent hosts requires them to be linked so more than one gene is incorporated into the genome at a time. The required linker regions can result in reduced functionality. In some cases, such as for bacterial luciferase, this significantly impairs functionality at 37° C., resulting in diminished light output under standard culture conditions. As a result, there have been no successful demonstrations of the stable generation of continuously or autonomously bioluminescent animal cells using any luminescent system with a known luciferin/luciferase pathway that functions efficiently at its optimal growth temperature.

Summary of the Technology: The disclosed method enables stable, multigene expression of luciferin/luciferase pathway genes for thermostable protein expression, allowing continuous or autonomous light production in the host. It may be used for small animal or cell-based research and development because it provides a means for non-invasively monitoring specific cells in real-time over prolonged time periods. The method comprises linking multiple luciferase and substrate processing genes using 2A linker regions containing integral protease recognition sites. Although there are a multitude of different strategies for multigene co-expression (e.g., expression as multiple open reading frames with individual promoters, fusion with linking amino acid chains, or IRES elements), it was found that only 2A elements permitted reliable multigene expression in a format amenable to efficient transfection. Counter to the common knowledge in the field that increasing numbers of 2A-linked open reading frames reduces translational efficiency, it was discovered that sufficiently strong promoters could drive expression of at least six individual open reading frames as a single mRNA under this strategy. As expected, incorporation of 2A element linkers between open reading frames caused translation of individual proteins from the mRNA. Unexpectedly, the resulting proteins were highly inefficient at temperatures above 25° C. A variety of hypothesizes were explored before discovering the artifactual C-terminal 2A element amino acids were responsible for this inefficiency. This finding was unexpected not only because physically linked bacterial luciferase proteins have been demonstrated as functional at these temperatures, but also because it contradicted the consensus within the field that 2A linker sequences do not alter the functionality of the up- or downstream protein to which they are appended. Incorporation of a protease recognition site between the concluding amino acid residue of the upstream protein and the leading amino acid residue of the 2A linker allowed for removal of the artifactual C-terminal amino acids and the protease recognition site itself. Removal of these artifactual sequences restored functionality to the luciferase/luciferin system at temperatures above 25° C. and enabled it to be stably introduced into the cellular genome such that the host cell could continuously or autonomously produce a luminescent signal throughout its lifespan and pass that phenotype to all daughter cells. This discovery significantly improves the utility of cellular assays by providing a means for continuous, non-invasive monitoring of cells using bioluminescence.

FIG. 1 illustrates an overview of the system. Multiple open readings frames are connected by intervening protease recognition sequences and 2A linkers. This architecture can be repeated as many times as needed to encode the open reading frames necessary for the desired functionality.

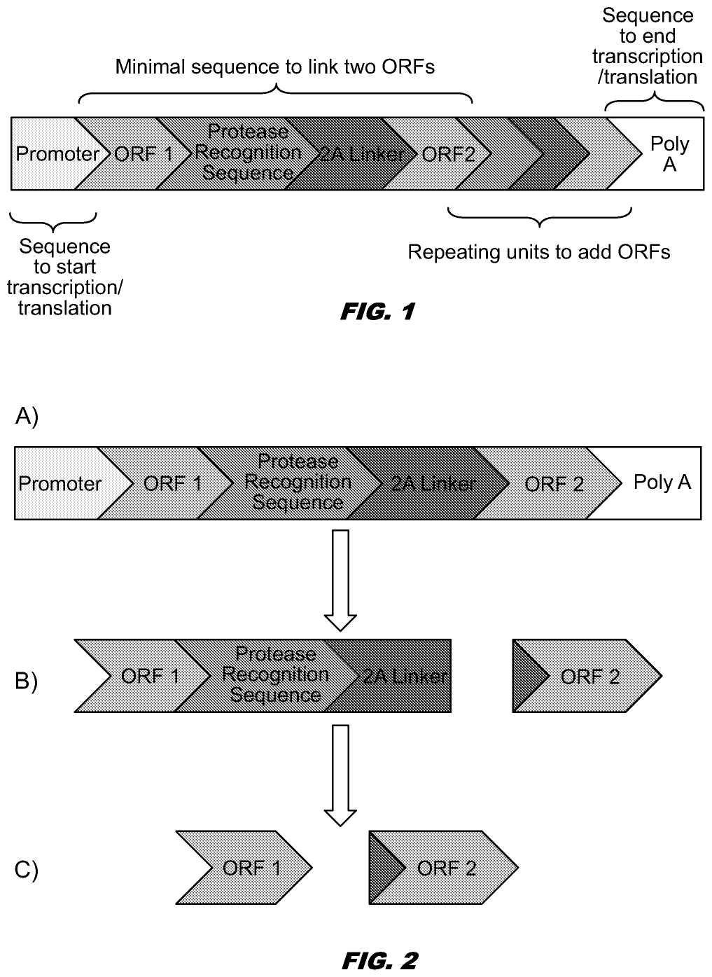

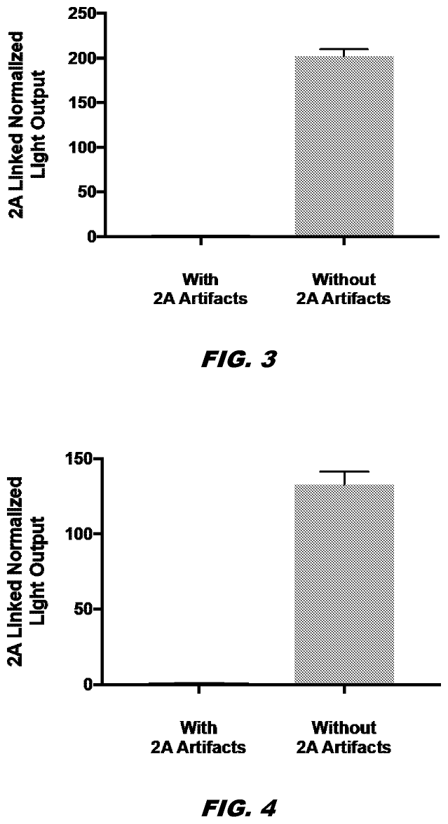

FIG. 2 illustrates the functionality of the system. A) The 2A elements allow a single encoded sequence to be transcribed and translated into B) individual proteins with artifactual amino acid residues from the protease recognition sites and 2A linkers attached. C) Endogenous proteases remove the artifactual amino acid residues, resulting in individual proteins that more closely match their native amino acid identity.

Description of Selected Embodiments: The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The described system provides advantages over previous systems and alternative approaches. Using linker regions resulting in independent proteins, rather than physically linked proteins or functional units, enables the resulting protein products to take advantage of intracellular environmental dynamics for access to intracellular materials and prevents interactional inhibition due to steric limitations. Furthermore, it allows multiple functional units to be delivered to a cell simultaneously, enables ratio-based introduction of DNA sequences for copy number control, and provides a facile method for coordinated regulation of subsets of the expressed cohort.

Relative to alternative approaches, using 2A linkers reduces the length of DNA that must be introduced and incorporated into the cellular genome to achieve pathway expression, which improves the efficiency of the transfection and selection processes. The variety of different 2A linker sequences available ensures that repetitive DNA sequence utilization, which can result in unintended natural modification within the host and increase the difficulty of genetic manipulation at the bench, can be avoided. For one skilled in the art, the differential efficiencies of available 2A linkers can also be used to modify transcriptional expression ratios of the linked open reading frames through rational design of the pathway expression order.

The use of protease recognition sequences provides a simplistic method for removing artifactual amino acid residues from the expressed proteins and therefore increasing the likelihood of wild type functionality. The advantages of the detailed Furin recognition sequences parallel those of the 2A linker regions. They can be encoded using a short DNA sequence, multiple DNA and amino acid identities are available to enable codon optimization while avoiding repetitive DNA sequences, and there is a large body of research available to inform sequence design relative to the surrounding amino acid residues in order to modulate efficiency. Also similar to the use of 2A linkers, they are non-coding sequences and function entirely using host machinery. This limits the number of exogenous genes that must be introduced to enable system functionality and therefore limits the impact of exogenous expression on the host.

In a basic embodiment, the system is can be comprised of repeating genetic structures in the form of an upstream open reading frame, a protease recognition site, a linker region, and a downstream open reading frame, as read in a 5′ to 3′ direction on a sense DNA strand. The downstream open reading frame then serves as the upstream open reading frame of any the further repetitions. In this fashion, any number of open reading frames can be linked together such that they produce individual proteins from a single mRNA, with the artifactual amino acids encoded by the protease recognition sequence and the linker region removed by an endogenous protease.

In some embodiments, spacer regions comprise additional nucleotide regions may be placed between any of the listed elements. These nucleotides can serve to encode additional functionalities, to target the mRNA or protein products to specific locations within the cell or extracellularly, to increase the distance between elements, to impart structures that modify the efficiency of the protease or ribosome at the DNA, RNA, or polypeptide level, to encourage or discourage epigenetic modification, or to encode flexible protein regions that modify the functionality or efficiency of the linker regions. These additional nucleotide regions may function to affect the upstream open reading frame, the downstream open reading frame, distal open reading frames, multiple open reading frames, none of the open reading frames, or any combination thereof.

In some embodiments, the additional nucleotide regions are incorporated into the adjacent open reading frame to function as part of the adjoining protein product. Examples of these include the addition of PEST sequences or other degradation tags to decrease protein half-life. In further embodiments the additional nucleotide regions can comprise binding or purification tags, for example polyhistidine tags or streptavidin or avidin fusion proteins. When placed between the open reading frame and the protease recognition site, the binding properties of these tags are unhindered by the presence of artifactual amino acids resulting from inclusion of the protease recognition sequence and linker region. In further embodiments, the additional nucleotide regions can encode recognitions sequences for DNA-binding proteins, polypeptides, enzymes, DNA, RNA, or non-organic substances.

In some embodiments, the additional nucleotide regions may contain nuclease recognition sequences, meganuclease recognition sequences, or unique nucleotide sequences that can at as barcodes, binding sites for CRISPER/Cas9, transcription activator-like effector nucleases (TALENs), or zinc finger nucleases, transposase recognition sites, viral insertion sites, or similar DNA modification systems. Inclusion of these sequences allows one skilled in the art to easily modify the pathway in question. For example, inserting additional open reading frames, adding or removing stop codons or other regulatory signals, or enabling/disabling alternative splicing of the mRNA.

In some embodiments, the linker regions used are 2A linker regions such as T2a, E2a, F2a, P2a, FMDV2a, or similar. For some linker regions, it may be necessary to introduce amino acid substitutions to the canonical linker sequence so that undesired protease recognitions motifs are avoided or secondary structures are not formed, to abolish potential binding interactions, or prevent similar unwanted functionality. For some linker regions, it may be desirable to leverage a naturally occurring protease recognition site within the linker either in place of, or in addition to, a separately incorporated protease recognition site. For some linker regions, it may not be necessary to impart any modifications. One skilled in the art can use the presence and/or absence of individual or multiple modifications to change the location and/or efficiency of the protease recognition sequence to fine tune its functionality within the system.

In some embodiments, the protease recognition sequences are Furin recognition sequences. In some embodiments the protease recognition sequences are, Enterokinase recognition sequences, Factor Xa recognition sequences, Subtilisin BPN″ recognition sequences, TEV recognition sequences, HRV 3C Protease recognition sequences, or similar. The recognition sequence for the employed protease can be chosen from among the full group of amino acid sequences recognized by the desired protease. Each possible amino acid recognition sequence for a given protease may have a different efficiency. One skilled in the art may leverage these efficiency differences to modify the functionality of the system. Similarly, one skilled in the art may select an amino acid sequence such that the residues present contribute in part or in full to function as an alternative functional sequence.

In some embodiments, upstream of the first open reading frame in the 5′ to 3′ direction on a sense DNA strand can be a promoter, enhancer, operator, or other element capable of initiating or regulating transcription or translation of the downstream open reading frames, or any combination thereof. In some embodiments, downstream of the last open reading frame in the 5′ to 3′ direction on a sense DNA strand can be one or more stop codons, a poly-A sequence, terminator, or other element capable of stopping transcription or translation of the encoded sequence, or any combination thereof.

In some embodiments, the full pathway of interest may be encoded as a single unit for coordinated expression of all pathway open reading frames simultaneously. In other embodiments the pathway of interest may be broken into subsections so that expression of each subsection can be controlled independently. In further embodiments, some or all of the pathway of interest may be expressed using these strategies while relying on traditional exogenous expression of one or more pathway components, or endogenous expression of necessary or equivalent pathway components from the host cell or the environment. One skilled in the art can use these strategies to control relative pathway or exogenous gene expression such that different ratios of transcribed or translated products are produced relative to native or exogenous genes.

In one example of functionality, the bacterial luciferase bioluminescent pathway was expressed in human cells using this system. The bacterial luciferase bioluminescent pathway presents an suitable example because it comprises multiple exogenous genes and does not function efficiently at the mammalian growth temperature optimum of 37° C. if stably expressed using traditional approaches. In fact, this approach is the only known method for enabling functional, stable expression of the bacterial luciferase bioluminescent pathway in human cells.

In this example, the bacterial luciferase pathway genes, luxC, luxD, luxA, luxB, and luxE, and a supporting oxidoreductase gene, frp, were codon optimized for expression in HEK293 cells. The stop codons were removed from the luxC, luxD, luxA, luxB, and luxE genes. A Furin protease recognition sequence (R-K-R-R), followed by a T2a 2A linker was placed between the luxC and luxD genes. A Furin protease recognition sequence (R-K-R-R), followed by a E2a 2A linker was placed between the luxD and luxA genes. A Furin protease recognition sequence (R-K-R-R), followed by a P2a 2A linker was placed between the luxA and luxB genes. A Furin protease recognition sequence (R-K-R-R), followed by a Pa2a 2A linker (comprising a P2a 2A linker amino acid sequence encoded by an alternative DNA sequence) was placed between the luxB and luxE genes. A Furin protease recognition sequence (R-K-R-R), followed by a FMDV 2A linker was placed between the luxE and frp genes. This full sequence was placed under the control of a CMV IE enhancer and CMV IE promoter and transfected into HEK293 cells. Autonomously bioluminescent isolates were selected based on light output and resistance to G418 as encoded by a selection marker on the delivery vector.

Stably selected cells developed using this method were capable of autonomously producing a bioluminescent signal when cultured at 37° C. This is a significantly different result than can be achieved using alternative strategies, such as expressing the bacterial luciferase genes from individual promoters, using IRES elements to express multiple bacterial luciferase genes, or linking bacterial luciferase genes with 2A linkers without protease recognition sequences; all of which fail to either stably express the bacterial luciferase bioluminescent pathway, or stably express the pathway but prevent efficient generation of a bioluminescent signal at 37° C.

As an alternative example, this strategy can be used to stably express the fungal luciferase bioluminescent pathway in eukaryotic cells. Like the bacterial luciferase bioluminescent pathway, the fungal luciferase bioluminescent pathway comprises multiple exogenous genes. However, in this example, the genes are sourced from multiple different organisms. In this example, a Rhodobacter capsulatus tyrosine ammonia lyase and two Escherichia coli 4-hydroxyphenylacetate 3-monooxygenase components are linked with the fungal genes npgA, hisps, h3h, and luz using intervening protease recognition sequences and 2A linkers. As before, this approach allows the individual open reading frames to be transcribed as a single mRNA, translated as individual proteins, and then processed by endogenous proteases such that the artifactual amino acids from the protease recognition and 2A linker sequences are removed.

This approach could also be applied to bioluminescent systems with more complex expression pathways, such as the luciferase pathways from fireflies, sea pansies, copepods, or dinoflagellates. Due to the complexity of these pathways, multiple strategies can be used. As one example, the full complement of genes required for luciferase, luciferin, and supporting analyte processing could be encoded as a single operon with intervening protease recognition sequences and 2A linkers. In another example, only those proteins without homologs in the host cell could be encoded as a single operon with intervening protease recognition sequences and 2A linkers, while the functions of the non-encoded open reading frames are performed by native homologs from the host cell. In another example, portions of the pathway are expressed individually, while other portions are encoded as a single operon with intervening protease recognition sequences and 2A linkers. In a further example, any combination of these strategies may be employed to achieve pathway functionality.

This approach is not limited to luciferase/luciferin pathway expression and can be used for thermostable expression of any multigene system. In a basic example, the approach can be used to express an upstream gene of interest with a downstream fluorescent reporter gene, such as GFP, YFP, RFP, mOrange, mCherry, dsRed, or similar. This configuration allows thermostable expression of the upstream gene of interest in its native form and expression of the downstream reporter protein to positively identify cells actively transcribing and translating the gene of interest and/or quantify transcriptional/translational levels of the gene of interest by measuring the fluorescent output of the downstream reporter. In a more complicated extension of this example, multiple genes of interest can be linked upstream of a reporter gene to enable similar capabilities with a more complex pathway. In some embodiments of this example, multiple fluorescent reporter genes can be interspersed among the genes of interest to enable estimation of the transcriptional/translational levels of one or more genes along the pathway.

In an embodiment of this example, the approach was used to restore correct protein targeting by obviating the disruption of signal proteins resulting from association with 2A linkers. For example, when a fluorescent reporter gene, dsRed, with a C-terminal peroxisome targeting sequence was placed upstream of a second fluorescent reporter gene, GFP, without a targeting sequence using a 2A linker, the dsRed protein failed to localize to the peroxisome and was expressed cytosolically similarly to the untagged GFP protein because the presence of the artifactual amino acids from the 2A linker modified the C-terminus of the protein such that the peroxisome targeting sequence could no longer be recognized by its receptor protein. However, when an intervening protease recognition sequence was added upstream of the 2A linker, protease cleavage removed the artifactual amino acids and restored the correct positioning of the peroxisome targeting sequence. As a result, functionality was restored and the dsRed protein was correctly trafficked to the peroxisome.

In other embodiments of this example, the reporter gene could be substituted for an antibiotic resistance gene. Placing the antibiotic resistance gene downstream of the gene(s) of interest with an intervening protease recognition sequence and 2A linker allows thermostable expression of the gene(s) of interest in their native forms and expression of the antibiotic resistance protein allows one to positively identify cells actively transcribing and translating the gene(s) of interest and/or stably selection and propagation of clonal lineages of those cells. In other embodiments, the gene(s) encoding antibiotic resistance may be expressed separately from the genes of interest.

In a further example outside of luciferase/luciferin pathway expression, the system could be used to simultaneously express thermostable versions of the four Yamanaka reprogramming factor genes: Oct-4, Sox2, Klf4, and c-Myc as a single operon with intervening protease recognition sequences and 2A linkers. This approach is advantageous relative to alternative approaches in that all four of the genes could be placed under the control of an inducible promoter to enable precise control over expressional timing. The ability to stably express thermostable versions of these proteins with a single point of control is advantageous for regenerative medicine, developmental biology, cellular biology, and basic research, and related fields of study.

The system may also have clinical or therapeutic applications. In clinical or therapeutic applications, it is paramount that proteins be expressed in their native form or without unintended modifications to their desired form. Furthermore, deployment of gene therapies within human subjects requires that the employed protein products remain thermostable and are expressed in a controlled fashion. The use of this system of open reading frames interspersed with intervening protease recognition sequences and 2A linkers allows these criteria to be met. In a generalized example of this application, a patient deficient in the expression of multiple genes could be treated with DNA or RNA encoding the deficient gene products. Upon translation, the presence of intervening protease recognition sequences and 2A linkers among the open reading frames would result in thermostable versions of the target proteins without artifactual amino acids that could modify their functionality or longevity. Furthermore, as described in previous examples, one skilled in the art could leverage the relative orientation of these ORFs, the presence or absence of additional nucleotide regions, combinations of these factors, or other similar control strategies to fine tune the transcription and/or translation of the nucleotides to improve therapeutic outcomes.

These disclosed embodiments are illustrative, not restrictive. While specific configurations of the expression system have been described, it is understood that the present invention can be applied to a wide variety of biotechnology applications. There are many alternative ways of implementing the invention.

### Example 2

As shown in FIG. 3, linking luciferin/luciferase pathway genes using 2A elements results in decreased performance compared to expression without the artifactual amino acids that remain following translation of individual proteins. When expressed in the same host cell, a 203 (±7) fold increase in light production was observed using an expression strategy that did not contain artifactual amino acid residues from 2A linker regions between genes.

### Example 3

As shown in FIG. 4, bioluminescent production is significantly improved at 37° C. by including Furin recognition sites upstream of viral 2A linkers between human codon optimized bacterial luciferase genes in HEK293 cells. Incorporating Furin recognition sites and removing artifactual amino acids that would normally remain after 2A linker functionality resulted in a 133 (±9) fold increase in light output compared to using only 2A linkers and retaining the artifactual amino acid sequences at the C-terminus of the luciferin/luciferase genes.

### Example 4

Basic Methods

The pCMVₗᵤₓ vector, which contains the LuxC, luxD, luxA, luxB, luxE, and frp genes required for autonomous bioluminescent production linked by viral 2A element spacers, was used as the basis for developing an improved vector with self-cleaving, 2A-linked sequences. The bacterial luciferase/luciferin cassette portion of the vector sequence was modified in silico to incorporate protease recognition sequences between each gene and its downstream viral 2A linker sequence. These sequence files were then broken into fragments consistent with the length limitations of DNA synthesis to represent the luxC-linker-luxD-linker, luxA-linker-luxB, and linker-luxE-linker-frp fragments. Overlapping sequences consisting of a minimum of 20 nucleodites were incorporated at the ends of each segment. The custom designed DNA sequences were synthesized and obtained as double stranded DNA. The pCMVlux vector was restriction digested to remove the previous cassette sequence lacking protease recognition sequences and the backbone was purified. The individual segments were then linked together and assembled into the pCMVₗᵤₓ backbone in place of the original 2A-linked luciferin/luciferase pathway cassette using a HiFi DNA Assembly reaction.

HEK-293 cells were cultured in a humidified incubator at 37° C. with 5% CO₂ and grown in Dulbecco's Modified Eagle Medium (DMEM) supplemented with 10% Fetal Bovine Serum (FBS), 1×Penn/Strep (ThermoFisher), and 1×GlutaMAX (ThermoFisher). HEKs were plated at 10,000 cells/well in a 96 well plate 24 hours prior to transfection. Transfection mixes were prepared by combining 100 ng of DNA with Viafect Transfection Reagent. The transfection mixes were incubated at room temperature for 10 minutes then added dropwise to HEKs. During the transfection process, the cells were housed in the humidified incubator at 37° C. with 5% CO₂. BMG LABTECH's CLARIOstar was used to analyze transfected HEKs at 24 and 48 hours post transfection using 1 second integration at 25 or 37° C. The total light production was quantified from each well and compared to mock transfected controls to determine the success of the transfection and the performance of the improved expression cassette.

### LISTING OF EXEMPLARY EMBODIMENTS

Embodiment 1: A nucleic acid construct configured to encode at least two genes of a multigene pathway in a cell, the nucleic acid construct comprising:
a plurality of nucleic acid sequences, wherein the plurality of nucleic acid sequences comprises:
   a first nucleic acid sequence encoding at least one gene of the multigene pathway;
   a first protease recognition nucleic acid sequence encoding a protease recognition site;
   a first linker nucleic acid sequence encoding a linker region, wherein the linker region comprises a viral 2A peptide; and
   a second nucleic acid sequence encoding at least one gene of the multigene pathway,
   wherein the first nucleic acid sequence and the second nucleic acid sequence are joined via the first linker nucleic acid sequence, and the first protease recognition nucleic acid sequence is located between the first nucleic acid sequence and the first linker nucleic acid sequence.

Embodiment 2: The nucleic acid construct of embodiment 1, wherein one or more of the plurality of nucleic acid sequences are adjacent and bonded to one another via a phosphodiester bond, a phosphorothionate bond, or a combination thereof.

Embodiment 3: The nucleic acid construct of embodiment 1, wherein the multigene pathway is thermostable at a cell culture relevant temperature.

Embodiment 4: The nucleic acid construct of embodiment 1, wherein:
the first nucleic acid sequence comprises a first luciferin/luciferase nucleic acid sequence;
the second nucleic acid sequence comprises a second luciferin/luciferase nucleic acid sequence; and
the multigene pathway comprises a luciferin/luciferase pathway.

Embodiment 5: The nucleic acid construct of embodiment 4, wherein the first luciferin/luciferase nucleic acid sequence and the second luciferin/luciferase nucleic acid sequence are configured to encode different genes of the luciferin/luciferase pathway.

Embodiment 6: The nucleic acid construct of embodiment 4, wherein the plurality of nucleic acid sequences further comprises:
a third nucleic acid sequence encoding an oxidoreductase gene;
a second protease recognition nucleic acid sequence encoding a second protease recognition site; and
a second linker nucleic acid sequence encoding a second linker region, wherein the second linker region comprises a viral 2A peptide,
wherein the second nucleic acid sequence and the third nucleic acid sequence are joined via the second linker nucleic acid sequence, and the second protease recognition nucleic acid sequence is located between 5 the second nucleic acid sequence and the second linker nucleic acid sequence.

Embodiment 7: The nucleic acid construct of embodiment 6, wherein the oxidoreductase gene comprises frp.

Embodiment 8: The nucleic acid construct of embodiment 4, wherein the luciferin/luciferase pathway comprises a bacterial luciferin/luciferase pathway, a fungal luciferin/luciferase pathway, or a combination thereof.

Embodiment 9: The nucleic acid construct of embodiment 4, wherein the first luciferin/luciferase nucleic acid sequence or the second luciferin/luciferase nucleic acid sequence encode for one or more of luxC, luxD, luxA, luxB, luxE, luxF, luxG, luxH, luxI, luxR, luxY, or frp.

Embodiment 10: The nucleic acid construct of embodiment 4, wherein the first luciferin/luciferase nucleic acid sequence or the second luciferin/luciferase nucleic acid sequence encode for one or more genes involved in synthesis of caffeic acid.

Embodiment 11: The nucleic acid construct of embodiment 10, wherein the one or more genes involved in the synthesis of caffeic acid comprise: a tyrosine ammonia lyase, two 4-hydroxyphenylacetate 3-monooxygenase components, a 4′-phosphopantetheinyl transferase, or a combination thereof.

Embodiment 12: The nucleic acid construct of embodiment 4, wherein the first luciferin/luciferase nucleic acid sequence or the second luciferin/luciferase nucleic acid sequence encode for luz, H3H, or HipS.

Embodiment 13: The nucleic acid construct of embodiment 4, comprising at least six luciferin/luciferase nucleic acid sequences, wherein each of the at least six luciferin/luciferase nucleic acid sequences encodes for a different gene of the luciferin/luciferase pathway.

Embodiment 14: The nucleic acid construct of embodiment 13, wherein the different genes of the luciferin/luciferase pathway comprise luxC, luxD, luxA, luxB, luxE, and frp.

Embodiment 15: The nucleic acid construct of embodiment 4, wherein the first luciferin/luciferase nucleic acid sequence or the second luciferin/luciferase nucleic acid sequence is at least 90% identical to SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, or SEQ ID NO: 47.

Embodiment 16: The nucleic acid construct of embodiment 4, wherein the first luciferin/luciferase nucleic acid sequence or the second luciferin/luciferase nucleic acid sequence encode for an amino acid sequence that is at least 90% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15.

Embodiment 17: The nucleic acid construct of embodiment 4, wherein at least one of the plurality of nucleic acid sequences encodes a gene for a luciferase enzyme.

Embodiment 18: The nucleic acid construct of embodiment 4, wherein at least one of the plurality of nucleic acid sequences encodes a gene for a protein required for luciferin substrate production.

Embodiment 19: The nucleic acid construct of embodiment 1, wherein the protease recognition site comprises a recognition site for furin.

Embodiment 20: The nucleic acid construct of embodiment 1, wherein the protease recognition nucleic acid sequence is configured to encode an amino acid sequence comprising R-X-X-R.

Embodiment 21: The nucleic acid construct of embodiment 20, wherein the protease recognition nucleic acid sequence is configured to encode an amino acid sequence comprising R-K-R-R.

Embodiment 22: The nucleic acid construct of embodiment 1, wherein the viral 2A peptide comprises T2a, E2a, F2a, P2a, Pa2a, FMDV2a, or a combination thereof.

Embodiment 23: The nucleic acid construct of embodiment 1, wherein the first linker nucleic acid sequence is configured to encode an amino acid sequence comprising at least 90% identity to SEQ ID NO: 28, SEQ ID NO: 29, SEQ ID NO: 30, SEQ ID NO: 31, SEQ ID NO: 32, or a combination thereof.

Embodiment 24: The nucleic acid construct of embodiment 23, wherein the first linker nucleic acid sequence comprises at least 90% identity to SEQ ID NO: 60, SEQ ID NO: 61, SEQ ID NO: 62, SEQ ID NO: 63, SEQ ID NO: 64, or a combination thereof.

Embodiment 25: The nucleic acid construct of embodiment 1, further comprising at least one spacer region between one or more of the plurality of nucleic acid sequences, wherein the at least one spacer region comprises a plurality of nucleotides configured to: target mRNA or protein products to specific locations within the cell or extracellularly; increase the distance between one or more of the plurality of nucleic acid sequences; impart structures that modify the efficiency of a protease or a ribosome at the DNA, RNA, or polypeptide level; encode at least one flexible protein region to modify a functionality or an efficiency of the linker region; or a combination thereof.

Embodiment 26: The nucleic acid construct of embodiment 1, further comprising a promoter, an enhancer, an operator, or other element capable of initiating or regulating transcription or translation of one or more of the plurality of nucleic acid sequences.

Embodiment 27: The nucleic acid construct of embodiment 1, further comprising at least one stop codon, a poly-A sequence, a terminator, or other element capable of stopping transcription or translation of one or more of the plurality of nucleic acid sequences.

Embodiment 28: A vector comprising the nucleic acid construct of any one of embodiments 1-27.

Embodiment 29: A cell comprising the vector of embodiment 28.

Embodiment 30: A method of producing bioluminescence in a cell line, comprising:
introducing the nucleic acid construct of any one of embodiments 1-27 into a plurality of cells to form a plurality of transfected cells;
expressing the nucleic acid construct in the plurality of transfected cells; and
maintaining the plurality of transfected cells in a culture media and at a cell culture relevant temperature.

Embodiment 31: A method of forming an autonomously bioluminescent cell line, comprising: isolating one or more of the plurality of transfected cells of embodiment 30 to form an autonomously bioluminescent cell line.

Embodiment 32: The method of embodiment 30 or embodiment 31, wherein the cell culture relevant temperature comprises a temperature of at least 4° C.

Embodiment 33: A system for expression of bioluminescence in cells, the system comprising:
a cell line comprising the nucleic acid construct of any one of embodiments 1-27, the nucleic acid construct having a luciferase/luciferin pathway functional at temperatures used in generating cell cultures, growing cell cultures, maintaining cell cultures, or a combination thereof.

Embodiment 34: The system of embodiment 33, wherein the temperatures used in generating cell cultures, growing cell cultures, maintaining cell cultures, or a combination thereof comprise temperatures of greater than 4° C.

Embodiment 35: The system of embodiment 33, wherein the temperatures used in generating cell lines, growing cell cultures, maintaining cell cultures, or a combination thereof comprise temperatures of up to 60° C.

Embodiment 36: The system of embodiment 33, wherein the temperatures used in generating cell cultures, growing cell cultures, maintaining cell cultures, or a combination thereof comprise temperatures of about 37° C.

Embodiment 37: The system of embodiment 33, wherein the cell line comprises eukaryotic cells.

Embodiment 38: A system for co-expression of at least two functional luciferase/luciferin pathway genes in a cell, the system comprising:
a first luciferase/luciferin pathway gene, wherein the first luciferase/luciferin pathway gene is transfected into a cell; and
a second luciferase/luciferin pathway gene transfected into the cell,
   wherein the first and second luciferase/luciferin pathway genes are disposed within a single nucleic acid construct and form a luciferase/luciferin pathway capable of autonomously producing bioluminescence in the cell at cell culture relevant temperatures.

Embodiment 39: The method of embodiment 38, wherein the cell culture relevant temperatures comprise a temperature of at least 4° C.

Embodiment 40: The system of embodiment 38, wherein the cell culture relevant temperatures comprise temperatures up to 60° C.

Embodiment 41: The system of embodiment 38, wherein the cell culture relevant temperatures comprise temperatures of about 37° C.

Embodiment 42: The system of embodiment 38, wherein the cell line comprises eukaryotic cells.

Embodiment 43: A method of non-invasive cellular monitoring, the method comprising:
providing at least one cell producing bioluminescence, the cell having been transfected with the nucleic acid construct of any one of embodiments 1-27, wherein the bioluminescence is detectable at multiple time points and in real-time; and
monitoring the bioluminescence of the cell.

Embodiment 44: The method of embodiment 43, wherein the bioluminescence is detectable in the absence of an exogenous luminescent stimulator.

Embodiment 45: A nucleic acid cassette comprising components in the following structure, oriented in a 5′ to 3′ direction:

A-p-B-C₍ₙ₎, wherein
“A” comprises a nucleic acid sequence encoding at least one gene of a luciferase/luciferin pathway;
“p” comprises a nucleic acid sequence encoding a protease recognition site;
“B” comprises a nucleic acid sequence encoding a 2A peptide;
“C” comprises a nucleic acid sequence encoding at least one gene of a luciferase/luciferin pathway; and
“n” is the number of repetitions of the “-p-B-C” portion of the nucleic acid cassette.

Embodiment 46: The nucleic acid cassette of embodiment 45, wherein “-” comprises a phosphodiester bond, a phosphorothioate bond, or a combination thereof.

Embodiment 47: The nucleic acid cassette of embodiment 45, wherein
“n” comprises a first repetition and at least one additional repetition, and wherein
B, C, or both in the first repetition are not identical to B, C, or both, respectively, in the at least one additional repetition.

Embodiment 48: The nucleic acid cassette of embodiment 45, wherein “n” is 1, 2, 3, 4, 5, 6, 7, 8, 9, or 10.

Embodiment 49: The nucleic acid cassette of embodiment 45, wherein “n” is at least 10.

Embodiment 50: The nucleic acid cassette of embodiment 45, further comprising a localization signal or an excretion signal for targeted expression within a cell or for trafficking outside of a cell.

Embodiment 51: The nucleic acid cassette of embodiment 45, further comprising at least one sequence tag for isolation, identification, visualization, or a combination thereof of a cell having the nucleic acid cassette.

Embodiment 52: The nucleic acid cassette of embodiment 45, further comprising an element configured to initiate, enhance, regulate, or stop transcription or translation of A, p, B, C, or a combination thereof.

Embodiment 53: A vector comprising the nucleic acid cassette of any one of embodiments 45-52.

Embodiment 54: The vector of embodiment 53, wherein the vector is an expression vector.

Embodiment 55: A kit for producing a genetically engineered cell having autonomous luminescence, comprising:

a vector comprising the nucleic acid construct of any one of embodiments 1-27.

Embodiment 56: A method for producing a genetically engineered cell having autonomous luminescence, comprising:
transfecting a cell with a vector comprising the nucleic acid construct of any one of embodiments 1-27.

Embodiment 57: Any one of embodiments 55 or 56, wherein the genetically engineered cell is a stem cell.

Embodiment 58: Any one of embodiments 55-57, wherein the genetically engineered cell is a pluripotent stem cell, a mesenchymal stem cell, or a non-embryonic stem cell.

Embodiment 59: Any one of embodiments 55-58, wherein the genetically engineered cell luminesces in the absence of an exogenous luminescent stimulator.

Embodiment 60: Any one of embodiments 55-59, wherein the genetically engineered cell luminesces in the absence of a fluorescent stimulation signal or a chemical luminescent activator.

Embodiment 61: A method of real-time monitoring of cell population size of a genetically engineered cell having autonomous luminescence, comprising:
transfecting a cell with a vector comprising the nucleic acid construct of any one of embodiments 1-27 to produce the genetically engineered cell having autonomous luminescence;
measuring a luminescent signal emitted from the genetically engineered cell having autonomous luminescence; and
assessing the cell population size of the genetically engineered cell having autonomous luminescence based on the measured luminescent signal.

Embodiment 62: The method of embodiment 61, further comprising tracking the cell population size over two or more points in time.

Embodiment 63: A method of real-time monitoring of cell viability of a genetically engineered cell having autonomous luminescence, comprising:
transfecting a cell with a vector comprising the nucleic acid construct of any one of embodiments 1-27 to produce the genetically engineered cell having autonomous luminescence;
measuring a luminescent signal emitted from the genetically engineered cell having autonomous luminescence; and
assessing the cell viability of the genetically engineered cell having autonomous luminescence based on the measured luminescent signal.

Embodiment 64: The method of embodiment 63, further comprising tracking the cell viability over two or more points in time.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1.-64. (canceled)

65. A nucleic acid construct configured to encode at least two genes of a multigene pathway in a cell, the nucleic acid construct comprising:
a plurality of nucleic acid sequences, wherein the plurality of nucleic acid sequences comprises:
a first nucleic acid sequence encoding at least one gene of the multigene pathway;
a first protease recognition nucleic acid sequence encoding a protease recognition site;
a first linker nucleic acid sequence encoding a linker region, wherein the linker region comprises a viral 2A peptide; and
a second nucleic acid sequence encoding at least one gene of the multigene pathway, wherein the first nucleic acid sequence and the second nucleic acid sequence are joined via the first linker nucleic acid sequence, and the first protease recognition nucleic acid sequence is located between the first nucleic acid sequence and the first linker nucleic acid sequence.

66. The nucleic acid construct of claim 65, wherein:
the first nucleic acid sequence comprises a first luciferin/luciferase nucleic acid sequence;
the second nucleic acid sequence comprises a second luciferin/luciferase nucleic acid sequence; and
the multigene pathway comprises a luciferin/luciferase pathway.

67. The nucleic acid construct of claim 66, wherein the plurality of nucleic acid sequences further comprises:
a third nucleic acid sequence encoding an oxidoreductase gene;
a second protease recognition nucleic acid sequence encoding a second protease recognition site; and
a second linker nucleic acid sequence encoding a second linker region, wherein the second nucleic acid sequence and the third nucleic acid sequence are joined via the second linker nucleic acid sequence, and the second protease recognition nucleic acid sequence is located between the second nucleic acid sequence and the second linker nucleic acid sequence.

68. The nucleic acid construct of claim 66, wherein the luciferin/luciferase pathway comprises a bacterial luciferin/luciferase pathway, a fungal luciferin/luciferase pathway, or a combination thereof.

69. The nucleic acid construct of claim 66, wherein at least one of the plurality of nucleic acid sequences encodes a gene for a luciferase enzyme.

70. The nucleic acid construct of claim 66, wherein at least one of the plurality of nucleic acid sequences encodes a gene for a protein required for luciferin substrate production.

71. The nucleic acid construct of claim 65, wherein the protease recognition site comprises a recognition site for furin.

72. The nucleic acid construct of claim 65, wherein the viral 2A peptide comprises T2a, E2a, F2a, P2a, Pa2a, FMDV2a, or a combination thereof.

73. The nucleic acid construct of claim 65, further comprising at least one spacer region between one or more of the plurality of nucleic acid sequences, wherein the at least one spacer region comprises a plurality of nucleotides configured to:
target mRNA or protein products to specific locations within the cell or extracellularly;
increase the distance between one or more of the plurality of nucleic acid sequences;
impart structures that modify the efficiency of a protease or a ribosome at the DNA, RNA, or polypeptide level;
encode at least one flexible protein region to modify a functionality or an efficiency of the linker region;
or a combination thereof.

74. The nucleic acid construct of claim 65, further comprising a promoter, an enhancer, an operator, or other element capable of initiating or regulating transcription or translation of one or more of the plurality of nucleic acid sequences.

75. The nucleic acid construct of claim 65, further comprising at least one stop codon, a poly-A sequence, a terminator, or other element capable of stopping transcription or translation of one or more of the plurality of nucleic acid sequences.

76. A vector comprising the nucleic acid construct of claim 65.

77. A cell comprising the vector of claim 76.

78. A method of producing bioluminescence in a cell line, comprising:
introducing the nucleic acid construct of claim 65 into a plurality of cells to form a plurality of transfected cells;
expressing the nucleic acid construct in the plurality of transfected cells; and
maintaining the plurality of transfected cells in a culture media and at a cell culture relevant temperature.

79. A system for expression of bioluminescence in cells, the system comprising:
a cell line comprising the nucleic acid construct of claim 65, the nucleic acid construct having a luciferase/luciferin pathway functional at temperatures used in generating cell cultures, growing cell cultures, maintaining cell cultures, or a combination thereof.

80. A system for co-expression of at least two functional luciferase/luciferin pathway genes in a cell, the system comprising:
a first luciferase/luciferin pathway gene, wherein the first luciferase/luciferin pathway gene is transfected into a cell; and
a second luciferase/luciferin pathway gene transfected into the cell, wherein the first and second luciferase/luciferin pathway genes are disposed within a single nucleic acid construct and form a luciferase/luciferin pathway capable of autonomously producing bioluminescence in the cell at cell culture relevant temperatures.

81. A nucleic acid cassette comprising components in the following structure, oriented in a 5 ‘to 3’ direction: A-p-B-C(n), wherein:
“A” comprises a nucleic acid sequence encoding at least one gene of a luciferase/luciferin pathway;
“p” comprises a nucleic acid sequence encoding a protease recognition site;
“B” comprises a nucleic acid sequence encoding a 2A peptide;
“C” comprises a nucleic acid sequence encoding at least one gene of a luciferase/luciferin pathway; and
“n” is the number of repetitions of the “-p-B-C” portion of the nucleic acid cassette.

82. The nucleic acid cassette of claim 81, wherein “-” comprises a phosphodiester bond, a phosphorothioate bond, or a combination thereof.

83. The nucleic acid cassette of claim 81, wherein “n” comprises a first repetition and at least one additional repetition, and wherein B, C, or both in the first repetition are not identical to B, C, or both, respectively, in the at least one additional repetition.

84. The nucleic acid cassette of claim 81, further comprising a localization signal or an excretion signal for targeted expression within a cell or for trafficking outside of a cell.


SEQUENCE LISTING (74 sequences)

SEQ ID NO: 1 - protein, 480 aa, Photorhabdus luminescens
MTKKISFIINGQVEIFPESDDLVQSINFGDNSVYLPILNDSHVKNIIDCNGNNELRLHNIVNFLYTVGQRWKNEEYSRRRTYIRDLKKYMGYSEEMAKLEANWISMILCSKGGLYDVVENELGSRHIMDEWLPQDESYVRAFPKGKSVHLLAGNVPLSGIMSILRAILTKNQCIIKTSSTDPFTANALALSFIDVDPNHPITRSLSVIYWPHQGDTSLAKEIMRHADVIVAWGGPDAINWAVEHAPSYADVIKFGSKKSLCIIDNPVDLTSAATGAAHDVCFYDQRACFSAQNIYYMGNHYEEFKLALIEKLNLYAHILPNAKKDFDEKAAYSLVQKESLFAGLKVEVDIHQRWMIIESNAGVEFNQPLGRCVYLHHVDNIEQILPYVQKNKTQTISIFPWESSFKYRDALALKGAERIVEAGMNNIFRVGGSHDGMRPLQRLVTYISHERPSNYTAKDVAVEIEQTRFLEEDKFLVFVP

SEQ ID NO: 2 - protein, 307 aa, Photorhabdus luminescens
MENESKYKTIDHVICVEGNKKIHVWETLPEENSPKRKNAIIIASGFARRMDHFAGLAEYLSRNGFHVIRYDSLHHVGLSSGTIDEFTMSIGKQSLLAVVDWLTTRKINNFGMLASSLSARIAYASLSEINASFLITAVGVVNLRYSLERALGFDYLSLPINELPDNLDFEGHKLGAEVFARDCLDFGWEDLASTINNMMYLDIPFIAFTANNDNWVKQDEVITLLSNIRSNRCKIYSLLGSSHDLSENLVVLRNFYQSVTKAAIAMDNDHLDIDVDITEPSFEHLTIATVNERRMRIEIENQAISLS

SEQ ID NO: 3 - protein, 360 aa, Photorhabdus luminescens
MKFGNFLLTYQPPQFSQTEVMKRLVKLGRISEECGFDTVWLLEHHFTEFGLLGNPYVAAAYLLGATKKLNVGTAAIVLPTAHPVRQLEDVNLLDQMSKGRFRFGICRGLYNKDFRVFGTDMNNSRALAECWYGLIKNGMTEGYMEADNEHIKFHKVKVNPAAYSRGGAPVYVVAESASTTEWAAQFGLPMILSWIINTNEKKAQLELYNEVAQEYGHDIHNIDHCLSYITSVDHDSIKAKEICRKFLGHWYDSYVNATTIFDDSDQTRGYDFNKGQWRDFVLKGHKDTNRRIDYSYEINPVGTPQECIDIIQKDIDATGISNICCGFEANGTVDEIIASMKLFQSDVMPFLKEKQRSLLY

SEQ ID NO: 4 - protein, 327 aa, Photorhabdus luminescens
MKFGLFFLNFINSTTVQEQSIVRMQEITEYVDKLNFEQILVYENHFSDNGVVGAPLTVSGFLLGLTEKIKIGSLNHIITTHHPVAIAEEACLLDQLSEGRFILGFSDCEKKDEMHFFNRPVEYQQQLFEECYEIINDALTTGYCNPDNDFYSFPKISVNPHAYTPGGPRKYVTATSHHIVEWAAKKGIPLIFKWDDSNDVRYEYAERYKAVADKYDVDLSEIDHQLMILVNYNEDSNKAKQETRAFISDYVLEMHPNENFENKLEEIIAENAVGNYTECITAAKLAIEKCGAKSVLLSFEPMNDLMSQKNVINIVDDNIKKYHMEYT

SEQ ID NO: 5 - protein, 370 aa, Photorhabdus luminescens
MTSYVDKQEITASSEIDDLIFSSDPLVWSYDEQEKIRKKLVLDAFRNHYKHCREYRHYCQAHKVDDNITEIDDIPVFPTSVFKFTRLLTSQENEIESWFTSSGTNGLKSQVARDRLSIERLLGSVSYGMKYVGSWFDHQIELVNLGPDRFNAHNIWFKYVMSLVELLYPTTFTVTEERIDFVKTLNSLERIKNQGKDLCLIGSPYFIYLLCHYMKDKKISFSGDKSLYIITGGGWKSYEKESLKRDDFNHLLFDTFNLSDISQIRDIFNQVELNTCFFEDEMQRKHVPPWVYARALDPETLKPVPDGTPGLMSYMDASATSYPAFIVTDDVGIISREYGKYPGVLVEILRRVNTRTQKGCALSLTEAFDS

SEQ ID NO: 6 - protein, 240 aa, Artificial Sequence
  Synthetic construct
MNNTIEPILGHRSIRKFTSEPIASEQLQTILQSGLAASSSSMLQVVSIIRVTDTEKRKLLAQYAGNQTYVESAAEFLVFCIDYQRHATINPDVQADFTELTLIGAVDSGIMAQNCLLAAESMGLGGVYIGGLRNSAAQVDELLGLPKNTAILFGMCLGHPDQSPETKPRLPAHVIVHENQYQALNIDDVQAYDKTMQEYYASRTSNQKQSVWSQETAGKLAGESRPHILPYLNSKGLAKR

SEQ ID NO: 7 - protein, 228 aa, Photobacterium leiognathi
MTKWNYGVFFLNFYHVGQQEPSLTMSNALETLRIMDEDTSIYDVVAFSEHHIDKSYNDEMKLAPFVSLGKQIHVLATSPETVVKAAKYGMPLLFKWDDSQQKRIELLNHYQAAAAKFNVDITGVRHRLMLFVNVNDNPTQAKAELSIYLEDYLSYTQAETSIDEIINSNAAGNFDTCLHHVAEMAQGLNNKVDFLFCFESMKDQENKKSLMINFDKRVINYRKEHNLN

SEQ ID NO: 8 - protein, 236 aa, Vibrio fischeri
MIVDGRVSKIVLASIKNNIYKVFITVNSPIKFIAGQFVMVTINGKKCPFSIANCPTKNHEIELHIGSSNKDCSLDIIEYFVDALVEEVAIELDAPHGNAWLRSESNNPLLLIAGGTGLSYINSILTNCLNRNIPQDIYLYWGVKNSSLLYEDEELLELSLNNKNLHYIPVIEDKSEEWIGKKGTVLDAVMEDFTDLAHFDIYVCGPFMMAKTAKEKLIEEKKAKSEQMFADAFAYV

SEQ ID NO: 9 - protein, 230 aa, Vibrio harveyi
MSSTSLLDEFGTPVQRVERAIEALKNGLGVLLMDDEDRENEGDLIFSAQHLTEAQMALMIREGSGIVCLCLTEERANWLDLPPMVKDNCSKNQTAFTVSIEAKEGVTTGVSAKDRVTTVKTATYFDAQPEDLARPGHVFPLVAKTNGVLARRGHTEGTIDLMYLANLVPSGILCELTNPDGTMAKLPETIEFARRHGMPVLTIEDIVDYRTGIDLRNEYKSGLVREVSWS

SEQ ID NO: 10 - protein, 193 aa, Vibrio fischeri
MTIMIKKSDFLAIPSEEYKGILSLRYQVFKQRLEWDLVVENNLESDEYDNSNAEYIYACDDTENVSGCWRLLPTTGDYMLKSVFPELLGQQSAPKDPNIVELSRFAVGKNSSKINNSASEITMKQFEAIYKHAVSQGITEYVTVTSTAIERFLKRIKVPCHRIGDKEIHVLGDTKSVVLSMPINEQFKKAVLN

SEQ ID NO: 11 - protein, 250 aa, Vibrio fischeri
MKNINADDTYRIINKIKACRAYDINQCLSDMTKMVHCEYYLTLAIIYPHSMVKSDISILDNYPKKWRQYYDDANLIKYDPIVDYSNSNHSPINWNIFENNAVNKKSPNVIKEAKTSGLITGFSFPIHTANNGFGMLSFAHSEKDNYIDSLFLHACMNIPLIVPSLVDNYRKINIANNKSNNDLTKREKECLAWACEGKSSWDISKILGCSERTVTFHLTNAQMKLNTTNRCQSISKAILTGAIDCPYFKN

SEQ ID NO: 12 - protein, 194 aa, Vibrio fischeri
MFKGIVEGIGIIEKIDIYTDLDKYAIRFPENMLNGIKKESSIMFNGCFLTVTSVNSNIVWFDIFEKEARKLDTFREYKVGDRVNLGTFPKFGAASGGHILSARISCVASIIEIIENEDYQQMWIQIPENFTEFLIDKDYIAVDGISLTIDTIKNNQFFISLPLKIAQNTNMKWRKKGDKVNVELSNKINANQCW

SEQ ID NO: 13 - protein, 267 aa, Artificial Sequence
  Cloning vector pnnLuz-C1
MRINISLSSLFERLSKLSSRSIAITCGVVLASAIAFPIIRRDYQTFLEVGPSYAPQNFRGYIIVCVLSLFRQEQKGLAIYDRLPEKRRWLADLPFREGTRPSITSHIIQRQRTQLVDQEFATRELIDKVIPRVQARHTDKTFLSTSKFEFHAKAIFLLPSIPINDPLNIPSHDTVRRTKREIAHMHDYHDCTLHLALAAQDGKEVLKKGWGQRHPLAGPGVPGPPTEWTFLYAPRNEEEARVVEMIVEASIGYMTNDPAGKIVENAK

SEQ ID NO: 14 - protein, 422 aa, Artificial Sequence
  Cloning vector pnnH3H-C1
MASFENSLSVLIVGAGLGGLAAAIALRRQGHVVKIYDSSSFKAELGAGLAVPPNTLRSLQQLGCNTENLNGVDNLCFTAMGYDGSVGMMNNMTDYREAYGTSWIMVHRVDLHNELMRVALDPGGLGPPATLHLNHRVTFCDVDACTVTFTNGTTQSADLIVGADGIRSTIRRFVLEEDVTVPASGIVGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHASLPTLGQGFGMGLEDAVALGTLLPKGTTASQIETRLAVYEQLRKDRAEFVAAESYEEQYVPEMRGLYLRSKELRDRVMGYDIKVESEKVLETLLRSSNSA

SEQ ID NO: 15 - protein, 1698 aa, Artificial Sequence
  Cloning vector pnnHispS-C1
MNSSKNPPSTLLDVFLDTARNLDTASRNVLECGEHRWSYRELDTVSSALAQHLRYTVGLSPTVAVISENHPYILALMLAVWKLGGTFAPIDVHSPAELVAGMLNIVSPSCLVIPSSDVTNQTLACDLNIPVVAFHPHQSTIPELNKKYLTDSQISPDLPFPDPNRPALYLFTSSATSRSNLKCVPLTHTFILRNSLSKRAWCKRMRPETDFDGIRVLGWAPWSHVLAHMQDIGPLTLLNAGCYVFATTPSTYPTELKDDRDVISCAANAVMYKGVKSFACLPFVLGGLKALCESEPSVKAQLQVEERAQLLKSLQHMKILECGGAMLEVSVASWAIENRIPISIGIGMTETGGALFAGPVQAIQTGFSSEDKFIEDATYLLVKDDYESHAEEDINEGELVVKSRMLPRGYLGYNDPSFSVDDAGWVTFKTGDRYSVTPDGKFSWLGRNTDFIQMTSGETLDPRPIESLLCESSLISRACVIGDKFLNGPATAVCAIIELEPTTVEKGQAHSRDIARIFAPINRDLPPPLRIAWSHVLVLQPSEKIPMTKKGTIFRKKIEQVFGSALGGSSGDNSQATTDASVVRRDELSNTVKHIISRVLGVSDDELLWTLSFAELGMTSALATRIANELNEVLVGVNLPINACYIHVDLPSLSNAVYAKLAHLKLPDRTPEPRKAPVENPGGKEIVIVGQAFRLPGSINDVASLRDAFLARQASSIITEIPPDRWDHASFYPKDIRFNKAGLVDIANYDHSFFGLTATEALYLSPTMRLALEVSFEALENANIPVSQLKGSQTAVYVATTDDGFETLLNAEAGYDAYTRFYGTGRAASTASGRISYLLDVHGPSITVDTACSGGAVCIDQAIDYLQSSSAADTAIICASNTHCWPGSFMFLSAQGMVSSGGRCATFTTDADGYVPSEGAVAFILKTREAAMRDKDTILATIKATQISHNGRSQGLVAPNVNSQADLHRSLLQKAGLSPADIHFIEAHGTGTSLGDLSEIQAINDAYTSSQPRTAGPLIVSASKTVIGHTEPAGPLVGMLSVLNSFKEGAVPGLAHLTADNLNPALDCSSVPLLIPYQPVHLAAPKPHRAAVMSYGFSGTLGGIVLEAPDEERLEEEPPNDKPMLFVVSAKTHTALIEYLGRYLEFLLQANPQDFCDICYTSCVGREHYRYRFACVANDMEDLIGQLQKRLGSKVPPKPSYKRGALAFAFSGQGTQFRGMATELAKAYSGFRKIVSDLAKRASELSGHAIDRFLLAYDIGAENVAPDSEADQICIFVYQCSVLRWLQTMGIRPSAVIGHSLGEISASVAAGALSLDSALDLVISRARLLRSSTNAPAGMAAMSASQDEVVELIGKLDLDKANSLSVSVINGPQNTVVSGSSAAIESIVALAKGRKIKASALNINQAFHSPYVDSAVPGLRAWSEKHISSARPLQIPLYSTLLGAQVSEGQMLNPDHWVDHARKPVQFAQAATIMKESFTGVIIDIGPQVVAWSLLLSNGLTSVTALAAKRGRSQQVAFLSALADLYQDYGVVPDFVGLYAQQEDASRLKKTDILTYPFQRVRRYPSFIPSRRAPTHAHVQDEETLSSGSSTPTLENTDLDSGKESLMGPTRGLLRVDDLRDSIVSSVKDVLELKSNEDLDLSESLNALGMDSIMFAQLRKRIGEGLGLSVPMVFLSDAFSIGEMVSNLVEQAEASEDN

SEQ ID NO: 16 - protein, 238 aa, Artificial Sequence
  Synthetic construct
MSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 17 - protein, 239 aa, Artificial Sequence
  Cloning vector pCI-YFP
MVSKGEELFTGVVPILVELDGDVNGHKFSVSGEGEGDATYGKLTLKFICTTGKLPVPWPTLVTTFGYGLQCFARYPDHMKLHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 18 - protein, 225 aa, Artificial Sequence
  Cloning vector pHUGE-RedSeed
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 19 - protein, 238 aa, Artificial Sequence
  Cloning vector OX4509
MASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFLRSRPKKKRKVEDP

SEQ ID NO: 20 - protein, 236 aa, Artificial Sequence
  Synthetic construct
MVSKGEENNMAIIKEFMRFKVRMEGSVNGHEFEIEGEGEGRPYEGFQTAKLKVTKGGPLPFAWDILSPQFTYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYK

SEQ ID NO: 21 - protein, 236 aa, Artificial Sequence
  Cloning vector pNIGEL17
MLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 22 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 23 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 24 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 25 - protein, 439 aa, Mus musculus
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 26 - protein, 4 aa, Artificial Sequence
  Furin recognition sequence
  MISC_FEATURE: Xaa can be any naturally occurring amino acid
  misc_feature (2)..(3): Xaa can be any naturally occurring amino acid
RXXR

SEQ ID NO: 27 - protein, 3 aa, Artificial Sequence
  peroxisome targeting sequence
SKL

SEQ ID NO: 28 - protein, 18 aa, Artificial Sequence
  T2a linker sequence
EGRGSLLTCGDVEENPGP

SEQ ID NO: 29 - protein, 20 aa, Artificial Sequence
  E2a linker sequence
QCTNYALLKLAGDVESNPGP

SEQ ID NO: 30 - protein, 19 aa, Artificial Sequence
  P2a linker sequence
ATNFSLLKQAGDVEENPGP

SEQ ID NO: 31 - protein, 19 aa, Artificial Sequence
  Pa2a linker sequence
ATNFSLLKQAGDVEENPGP

SEQ ID NO: 32 - protein, 20 aa, Artificial Sequence
  FMDV2a linker sequence
QLLNFDLLKLAGDVESNPGP

SEQ ID NO: 33 - DNA, 1440 bp, Photorhabdus luminescens
atgaccaagaagatcagcttcatcatcaacggccaggtggagatcttccccgagagcgacgacctggtgcagagcatcaacttcggcgacaacagcgtgtacctgcccatcctgaacgacagccacgtgaagaacatcatcgactgcaacggcaacaacgagctgcgcctgcacaacatcgtgaacttcctgtacaccgtgggccagcgctggaagaacgaggagtacagccgccgccgcacctacatccgcgacctgaagaagtacatgggctacagcgaggagatggccaagctggaggccaactggatcagcatgatcctgtgcagcaagggcggcctgtacgacgtggtggagaacgagctgggcagccgccacatcatggacgagtggctgccccaggacgagagctacgtgcgcgccttccccaagggcaagagcgtgcacctgctggccggcaacgtgcccctgagcggcatcatgagcatcctgcgcgccatcctgaccaagaaccagtgcatcatcaagaccagcagcaccgaccccttcaccgccaacgccctggccctgagcttcatcgacgtggaccccaaccaccccatcacccgcagcctgagcgtgatctactggccccaccagggcgacaccagcctggccaaggagatcatgcgccacgccgacgtgatcgtggcctggggcggccccgacgccatcaactgggccgtggagcacgcccccagctacgccgacgtgatcaagttcggcagcaagaagagcctgtgcatcatcgacaaccccgtggacctgaccagcgccgccaccggcgccgcccacgacgtgtgcttctacgaccagcgcgcctgcttcagcgcccagaacatctactacatgggcaaccactacgaggagttcaagctggccctgatcgagaagctgaacctgtacgcccacatcctgcccaacgccaagaaggacttcgacgagaaggccgcctacagcctggtgcagaaggagagcctgttcgccggcctgaaggtggaggtggacatccaccagcgctggatgatcatcgagagcaacgccggcgtggagttcaaccagcccctgggccgctgcgtgtacctgcaccacgtggacaacatcgagcagatcctgccctacgtgcagaagaacaagacccagaccatcagcatcttcccctgggagagcagcttcaagtaccgcgacgccctggccctgaagggcgccgagcgcatcgtggaggccggcatgaacaacatcttccgcgtgggcggcagccacgacggcatgcgccccctgcagcgcctggtgacctacatcagccacgagcgccccagcaactacaccgccaaggacgtggccgtggagatcgagcagacccgcttcctggaggaggacaagttcctggtgttcgtgccc

SEQ ID NO: 34 - DNA, 921 bp, Photorhabdus luminescens
atggagaacgagagcaagtacaagaccatcgaccacgtgatctgcgtggagggcaacaagaagatccacgtgtgggagaccctgcccgaggagaacagccccaagcgcaagaacgccatcatcatcgccagcggcttcgcccgccgcatggaccacttcgccggcctggccgagtacctgagccgcaacggcttccacgtgatccgctacgacagcctgcaccacgtgggcctgagcagcggcaccatcgacgagttcaccatgagcatcggcaagcagagcctgctggccgtggtggactggctgaccacccgcaagatcaacaacttcggcatgctggccagcagcctgagcgcccgcatcgcctacgccagcctgagcgagatcaacgccagcttcctgatcaccgccgtgggcgtggtgaacctgcgctacagcctggagcgcgccctgggcttcgactacctgagcctgcccatcaacgagctgcccaacaacctggacttcgagggccacaagctgggcgccgaggtgttcgcccgcgactgcctggacttcggctgggaggacctggccagcaccatcaacaacatgatgtacctggacatccccttcatcgccttcaccgccaacaacgacaactgggtgaagcaggacgaggtgatcaccctgctgagcaacatccgcagcaaccgctgcaagatctacagcctgctgggcagcagccacgacctgagcgagaacctggtggtgctgcgcaacttctaccagagcgtgaccaaggccgccatcgcgatggacaacgaccacctggacatcgacgtggacatcaccgagcccagcttcgagcacctgaccatcgccaccgtgaacgagcgccgcatgcgcatcgagatcgagaaccaggccatcagcctgagc

SEQ ID NO: 35 - DNA, 1080 bp, Photorhabdus luminescens
atgaagttcggcaacttcctgctgacctaccagcccccccagttcagccagaccgaggtgatgaagcgcctggtgaagctgggccgcatcagcgaggagtgcggcttcgacaccgtgtggctgctggagcaccacttcaccgagttcggcctgctgggcaacccctacgtggccgccgcctacctgctgggcgccaccaagaagctgaacgtgggcaccgccgccatcgtgctgcccaccgcccaccccgtgcgccagctggaggacgtgaacctgctggaccagatgagcaagggccgcttccgcttcggcatctgccgcggcctgtacaacaaggacttccgcgtgttcggcaccgacatgaacaacagccgcgccctggccgagtgctggtacggcctgatcaagaacggcatgaccgagggctacatggaggccgacaacgagcacatcaagttccacaaggtgaaggtgaaccccgccgcctacagccgcggcggcgcccccgtgtacgtggtggccgagagcgccagcaccaccgagtgggccgcccagttcggcctgcccatgatcctgagctggatcatcaacaccaacgagaagaaggcccagctggagctgtacaacgaggtggcccaggagtacggccacgacatccacaacatcgaccactgcctgagctacatcaccagcgtggaccacgacagcatcaaggccaaggagatctgccgcaagttcctgggccactggtacgacagctacgtgaacgccaccaccatcttcgacgacagcgaccagacccgcggctacgacttcaacaagggccagtggcgcgacttcgtgctgaagggccacaaggacaccaaccgccgcatcgactacagctacgagatcaaccccgtgggcaccccccaggagtgcatcgacatcatccagaaggacatcgacgccaccggcatcagcaacatctgctgcggcttcgaggccaacggcaccgtggacgagatcatcgccagcatgaagctgttccagagcgacgtgatgcccttcctgaaggagaagcagcgcagcctgctgtac

SEQ ID NO: 36 - DNA, 981 bp, Photorhabdus luminescens
atgaagttcggcctgttcttcctgaacttcatcaacagcaccaccgtgcaggagcagagcatcgtgcgcatgcaggagatcaccgagtacgtggacaagctgaacttcgagcagatcctggtgtacgagaaccacttcagcgacaacggcgtggtgggcgcccccctgaccgtgagcggcttcctgctgggcctgaccgagaagatcaagatcggcagcctgaaccacatcatcaccacccaccaccccgtgcgcatcgccgaggaggcctgcctgctggaccagctgagcgagggccgcttcatcctgggcttcagcgactgcgagaagaaggacgagatgcacttcttcaaccgccccgtggagtaccagcagcagctgttcgaggagtgctacgagatcatcaacgacgccctgaccaccggctactgcaaccccgacaacgacttctacagcttccccaagatcagcgtgaacccccacgcctacacccccggcggcccccgcaagtacgtgaccgccaccagccaccacatcgtggagtgggccgccaagaagggcatccccctgatcttcaagtgggacgacagcaacgacgtgcgctacgagtacgccgagcgctacaaggccgtggccgacaagtacgacgtggacctgagcgagatcgaccaccagctgatgatcctggtgaactacaacgaggacagcaacaaggccaagcaggagacccgcgccttcatcagcgactacgtgctggagatgcaccccaacgagaacttcgagaacaagctggaggagatcatcgccgagaacgccgtgggcaactacaccgagtgcatcaccgccgccaagctggccatcgagaagtgcggcgccaagagcgtgctgctgagcttcgagcccatgaacgacctgatgagccagaagaacgtgatcaacatcgtggacgacaacatcaagaagtaccacatggagtacacc

SEQ ID NO: 37 - DNA, 1110 bp, Photorhabdus luminescens
atgaccagctacgtggacaagcaggagatcaccgccagcagcgagatcgacgacctgatcttcagcagcgaccccctggtgtggagctacgacgagcaggagaagatccgcaagaagctggtgctggacgccttccgcaaccactacaagcactgccgcgagtaccgccactactgccaggcccacaaggtggacgacaacatcaccgagatcgacgacatccccgtgttccccaccagcgtgttcaagttcacccgcctgctgaccagccaggagaacgagatcgagagctggttcaccagcagcggcaccaacggcctgaagagccaggtggcccgcgaccgcctgagcatcgagcgcctgctgggcagcgtgagctacggcatgaagtacgtgggcagctggttcgaccaccagatcgagctggtgaacctgggtcccgaccgcttcaacgcccacaacatctggttcaagtacgtgatgagcctggtggagctgctgtaccccaccaccttcaccgtgaccgaggagcgcatcgacttcgtgaagaccctgaacagcctggagcgcatcaagaaccagggcaaggacctgtgcctgatcggcagcccctacttcatctacctgctgtgccactacatgaaggacaagaagatcagcttcagcggcgacaagagcctgtacatcatcaccggcggcggctggaagagctacgagaaggagagcctgaagcgcgacgacttcaaccacctgctgttcgacaccttcaacctgagcgacatcagccagatccgcgacatcttcaaccaggtggagctgaacacctgcttcttcgaggacgagatgcagcgcaagcacgtgcccccctgggtgtacgcccgcgccctggaccccgagaccctgaagcccgtgcccgacggcacccccggcctgatgagctacatggacgccagcgccaccagctaccccgccttcatcgtgaccgacgacgtgggcatcatcagccgcgagtacggcaagtaccccggcgtgctggtggagatcctgcgccgcgtgaacacccgcacccagaagggctgcgccctgagcctgaccgaggccttcgacagc

SEQ ID NO: 38 - DNA, 774 bp, Artificial Sequence
  Synthetic construct
atggtgaagatccagcccatccccaccaccagccagggcagcctgttcatcatgaacagcaccatcgagaccatcctgggccaccgcagcatccgcaagttcaccagcgagcccatcgccagcgagcagctgcagaccatcctgcagagcggcctggccgccagcagcagcagcatgctgcaggtggtgagcatcatccgcgtgaccgacaccgagaagcgcaagctgctggcccagtacgccggcaaccagacctacgtggagagcgccgccgagttcctggtgttctgcatcgactaccagcgccacgccaccatcaaccccgacgtgcaggccgacttcaccgagctgaccctgatcggcgccgtggacagcggcatcatggcccagaactgcctgctggccgccgagagcatgggcctgggcggcgtgtacatcggcggcctgcgcaacagcgccgcccaggtggacgagctgctgggcctgcccaagaacaccgccatcctgttcggcatgtgcctgggccaccccgaccagagccccgagaccaagccccgcctgcccgcccacgtgatcgtgcacgagaaccagtaccaggccctgaacatcgacgacgtgcaggcctacgacaagaccatgcaggagtactacgccagccgcaccagcaaccagaagcagagcgtgtggagccaggagaccgccggcaagctggccggcgagagccgcccccacatcctgccctacctgaacagcaagggcctggccaagcgctga

SEQ ID NO: 39 - DNA, 684 bp, Photobacterium leiognathi
atgaccaaatggaactatggcgtgttttttctgaacttttatcatgtgggccagcaggaaccgagcctgaccatgagcaacgcgctggaaaccctgcgcattatggatgaagataccagcatttatgatgtggtggcgtttagcgaacatcatattgataaaagctataacgatgaaatgaaactggcgccgtttgtgagcctgggcaaacagattcatgtgctggcgaccagcccggaaaccgtggtgaaagcggcgaaatatggcatgccgctgctgtttaaatgggatgatagccagcagaaacgcattgaactgctgaaccattatcaggcggcggcggcgaaatttaacgtggatattaccggcgtgcgccatcgcctgatgctgtttgtgaacgtgaacgataacccgacccaggcgaaagcggaactgagcatttatctggaagattatctgagctatacccaggcggaaaccagcattgatgaaattattaacagcaacgcggcgggcaactttgatacctgcctgcatcatgtggcggaaatggcgcagggcctgaacaacaaagtggattttctgttttgctttgaaagcatgaaagatcaggaaaacaaaaaaagcctgatgattaactttgataaacgcgtgattaactatcgcaaagaacataacctgaac

SEQ ID NO: 40 - DNA, 708 bp, Vibrio fischeri
atgattgtggatggccgcgtgagcaaaattgtgctggcgagcattaaaaacaacatttataaagtgtttattaccgtgaacagcccgattaaatttattgcgggccagtttgtgatggtgaccattaacggcaaaaaatgcccgtttagcattgcgaactgcccgaccaaaaaccatgaaattgaactgcatattggcagcagcaacaaagattgcagcctggatattattgaatattttgtggatgcgctggtggaagaagtggcgattgaactggatgcgccgcatggcaacgcgtggctgcgcagcgaaagcaacaacccgctgctgctgattgcgggcggcaccggcctgagctatattaacagcattctgaccaactgcctgaaccgcaacattccgcaggatatttatctgtattggggcgtgaaaaacagcagcctgctgtatgaagatgaagaactgctggaactgagcctgaacaacaaaaacctgcattatattccggtgattgaagataaaagcgaagaatggattggcaaaaaaggcaccgtgctggatgcggtgatggaagattttaccgatctggcgcattttgatatttatgtgtgcggcccgtttatgatggcgaaaaccgcgaaagaaaaactgattgaagaaaaaaaagcgaaaagcgaacagatgtttgcggatgcgtttgcgtatgtg

SEQ ID NO: 41 - DNA, 690 bp, Vibrio harveyi
atgagcagcaccagcctgctggatgaatttggcaccccggtgcagcgcgtggaacgcgcgattgaagcgctgaaaaacggcctgggcgtgctgctgatggatgatgaagatcgcgaaaacgaaggcgatctgatttttagcgcgcagcatctgaccgaagcgcagatggcgctgatgattcgcgaaggcagcggcattgtgtgcctgtgcctgaccgaagaacgcgcgaactggctggatctgccgccgatggtgaaagataactgcagcaaaaaccagaccgcgtttaccgtgagcattgaagcgaaagaaggcgtgaccaccggcgtgagcgcgaaagatcgcgtgaccaccgtgaaaaccgcgacctattttgatgcgcagccggaagatctggcgcgcccgggccatgtgtttccgctggtggcgaaaaccaacggcgtgctggcgcgccgcggccataccgaaggcaccattgatctgatgtatctggcgaacctggtgccgagcggcattctgtgcgaactgaccaacccggatggcaccatggcgaaactgccggaaaccattgaatttgcgcgccgccatggcatgccggtgctgaccattgaagatattgtggattatcgcaccggcattgatctgcgcaacgaatataaaagcggcctggtgcgcgaagtgagctggagc

SEQ ID NO: 42 - DNA, 579 bp, Vibrio fischeri
atgaccattatgattaaaaaaagcgattttctggcgattccgagcgaagaatataaaggcattctgagcctgcgctatcaggtgtttaaacagcgcctggaatgggatctggtggtggaaaacaacctggaaagcgatgaatatgataacagcaacgcggaatatatttatgcgtgcgatgataccgaaaacgtgagcggctgctggcgcctgctgccgaccaccggcgattatatgctgaaaagcgtgtttccggaactgctgggccagcagagcgcgccgaaagatccgaacattgtggaactgagccgctttgcggtgggcaaaaacagcagcaaaattaacaacagcgcgagcgaaattaccatgaaacagtttgaagcgatttataaacatgcggtgagccagggcattaccgaatatgtgaccgtgaccagcaccgcgattgaacgctttctgaaacgcattaaagtgccgtgccatcgcattggcgataaagaaattcatgtgctgggcgataccaaaagcgtggtgctgagcatgccgattaacgaacagtttaaaaaagcggtgctgaac

SEQ ID NO: 43 - DNA, 750 bp, Vibrio fischeri
atgaaaaacattaacgcggatgatacctatcgcattattaacaaaattaaagcgtgccgcgcgtatgatattaaccagtgcctgagcgatatgaccaaaatggtgcattgcgaatattatctgaccctggcgattatttatccgcatagcatggtgaaaagcgatattagcattctggataactatccgaaaaaatggcgccagtattatgatgatgcgaacctgattaaatatgatccgattgtggattatagcaacagcaaccatagcccgattaactggaacatttttgaaaacaacgcggtgaacaaaaaaagcccgaacgtgattaaagaagcgaaaaccagcggcctgattaccggctttagctttccgattcataccgcgaacaacggctttggcatgctgagctttgcgcatagcgaaaaagataactatattgatagcctgtttctgcatgcgtgcatgaacattccgctgattgtgccgagcctggtggataactatcgcaaaattaacattgcgaacaacaaaagcaacaacgatctgaccaaacgcgaaaaagaatgcctggcgtgggcgtgcgaaggcaaaagcagctgggatattagcaaaattctgggctgcagcgaacgcaccgtgacctttcatctgaccaacgcgcagatgaaactgaacaccaccaaccgctgccagagcattagcaaagcgattctgaccggcgcgattgattgcccgtattttaaaaac

SEQ ID NO: 44 - DNA, 582 bp, Vibrio fischeri
atgtttaaaggcattgtggaaggcattggcattattgaaaaaattgatatttataccgatctggataaatatgcgattcgctttccggaaaacatgctgaacggcattaaaaaagaaagcagcattatgtttaacggctgctttctgaccgtgaccagcgtgaacagcaacattgtgtggtttgatatttttgaaaaagaagcgcgcaaactggatacctttcgcgaatataaagtgggcgatcgcgtgaacctgggcacctttccgaaatttggcgcggcgagcggcggccatattctgagcgcgcgcattagctgcgtggcgagcattattgaaattattgaaaacgaagattatcagcagatgtggattcagattccggaaaactttaccgaatttctgattgataaagattatattgcggtggatggcattagcctgaccattgataccattaaaaacaaccagttttttattagcctgccgctgaaaattgcgcagaacaccaacatgaaatggcgcaaaaaaggcgataaagtgaacgtggaactgagcaacaaaattaacgcgaaccagtgctgg

SEQ ID NO: 45 - DNA, 801 bp, Artificial Sequence
  Cloning vector pnnLuz-C1
atgcgcattaacattagcctgagcagcctgtttgaacgcctgagcaaactgagcagccgcagcattgcgattacctgcggcgtggtgctggcgagcgcgattgcgtttccgattattcgccgcgattatcagacctttctggaagtgggcccgagctatgcgccgcagaactttcgcggctatattattgtgtgcgtgctgagcctgtttcgccaggaacagaaaggcctggcgatttatgatcgcctgccggaaaaacgccgctggctggcggatctgccgtttcgcgaaggcacccgcccgagcattaccagccatattattcagcgccagcgcacccagctggtggatcaggaatttgcgacccgcgaactgattgataaagtgattccgcgcgtgcaggcgcgccataccgataaaacctttctgagcaccagcaaatttgaatttcatgcgaaagcgatttttctgctgccgagcattccgattaacgatccgctgaacattccgagccatgataccgtgcgccgcaccaaacgcgaaattgcgcatatgcatgattatcatgattgcaccctgcatctggcgctggcggcgcaggatggcaaagaagtgctgaaaaaaggctggggccagcgccatccgctggcgggcccgggcgtgccgggcccgccgaccgaatggacctttctgtatgcgccgcgcaacgaagaagaagcgcgcgtggtggaaatgattgtggaagcgagcattggctatatgaccaacgatccggcgggcaaaattgtggaaaacgcgaaa

SEQ ID NO: 46 - DNA, 1266 bp, Artificial Sequence
  Cloning vector pnnH3H-C1
atggcgagctttgaaaacagcctgagcgtgctgattgtgggcgcgggcctgggcggcctggcggcggcgattgcgctgcgccgccagggccatgtggtgaaaatttatgatagcagcagctttaaagcggaactgggcgcgggcctggcggtgccgccgaacaccctgcgcagcctgcagcagctgggctgcaacaccgaaaacctgaacggcgtggataacctgtgctttaccgcgatgggctatgatggcagcgtgggcatgatgaacaacatgaccgattatcgcgaagcgtatggcaccagctggattatggtgcatcgcgtggatctgcataacgaactgatgcgcgtggcgctggatccgggcggcctgggcccgccggcgaccctgcatctgaaccatcgcgtgaccttttgcgatgtggatgcgtgcaccgtgacctttaccaacggcaccacccagagcgcggatctgattgtgggcgcggatggcattcgcagcaccattcgccgctttgtgctggaagaagatgtgaccgtgccggcgagcggcattgtgggctttcgctggctggtgcaggcggatgcgctggatccgtatccggaactggattggattgtgaaaaaaccgccgctgggcgcgcgcctgattagcaccccgcagaacccgcagagcggcgtgggcctggcggatcgccgcaccattattatttatgcgtgccgcggcggcaccatggtgaacgtgctggcggtgcatgatgatgaacgcgatcagaacaccgcggattggagcgtgccggcgagcaaagatgatctgtttcgcgtgtttcatgattatcatccgcgctttcgccgcctgctggaactggcgcaggatattaacctgtggcagatgcgcgtggtgccggtgctgaaaaaatgggtgaacaaacgcgtgtgcctgctgggcgatgcggcgcatgcgagcctgccgaccctgggccagggctttggcatgggcctggaagatgcggtggcgctgggcaccctgctgccgaaaggcaccaccgcgagccagattgaaacccgcctggcggtgtatgaacagctgcgcaaagatcgcgcggaatttgtggcggcggaaagctatgaagaacagtatgtgccggaaatgcgcggcctgtatctgcgcagcaaagaactgcgcgatcgcgtgatgggctatgatattaaagtggaaagcgaaaaagtgctggaaaccctgctgcgcagcagcaacagcgcg

SEQ ID NO: 47 - DNA, 5094 bp, Artificial Sequence
  Cloning vector pnnHispS-C1
atgaacagcagcaaaaacccgccgagcaccctgctggatgtgtttctggataccgcgcgcaacctggataccgcgagccgcaacgtgctggaatgcggcgaacatcgctggagctatcgcgaactggataccgtgagcagcgcgctggcgcagcatctgcgctataccgtgggcctgagcccgaccgtggcggtgattagcgaaaaccatccgtatattctggcgctgatgctggcggtgtggaaactgggcggcacctttgcgccgattgatgtgcatagcccggcggaactggtggcgggcatgctgaacattgtgagcccgagctgcctggtgattccgagcagcgatgtgaccaaccagaccctggcgtgcgatctgaacattccggtggtggcgtttcatccgcatcagagcaccattccggaactgaacaaaaaatatctgaccgatagccagattagcccggatctgccgtttccggatccgaaccgcccggcgctgtatctgtttaccagcagcgcgaccagccgcagcaacctgaaatgcgtgccgctgacccatacctttattctgcgcaacagcctgagcaaacgcgcgtggtgcaaacgcatgcgcccggaaaccgattttgatggcattcgcgtgctgggctgggcgccgtggagccatgtgctggcgcatatgcaggatattggcccgctgaccctgctgaacgcgggctgctatgtgtttgcgaccaccccgagcacctatccgaccgaactgaaagatgatcgcgatgtgattagctgcgcggcgaacgcggtgatgtataaaggcgtgaaaagctttgcgtgcctgccgtttgtgctgggcggcctgaaagcgctgtgcgaaagcgaaccgagcgtgaaagcgcagctgcaggtggaagaacgcgcgcagctgctgaaaagcctgcagcatatgaaaattctggaatgcggcggcgcgatgctggaagtgagcgtggcgagctgggcgattgaaaaccgcattccgattagcattggcattggcatgaccgaaaccggcggcgcgctgtttgcgggcccggtgcaggcgattcagaccggctttagcagcgaagataaatttattgaagatgcgacctatctgctggtgaaagatgattatgaaagccatgcggaagaagatattaacgaaggcgaactggtggtgaaaagccgcatgctgccgcgcggctatctgggctataacgatccgagctttagcgtggatgatgcgggctgggtgacctttaaaaccggcgatcgctatagcgtgaccccggatggcaaatttagctggctgggccgcaacaccgattttattcagatgaccagcggcgaaaccctggatccgcgcccgattgaaagcctgctgtgcgaaagcagcctgattagccgcgcgtgcgtgattggcgataaatttctgaacggcccggcgaccgcggtgtgcgcgattattgaactggaaccgaccaccgtggaaaaaggccaggcgcatagccgcgatattgcgcgcatttttgcgccgattaaccgcgatctgccgccgccgctgcgcattgcgtggagccatgtgctggtgctgcagccgagcgaaaaaattccgatgaccaaaaaaggcaccatttttcgcaaaaaaattgaacaggtgtttggcagcgcgctgggcggcagcagcggcgataacagccaggcgaccaccgatgcgagcgtggtgcgccgcgatgaactgagcaacaccgtgaaacatattattagccgcgtgctgggcgtgagcgatgatgaactgctgtggaccctgagctttgcggaactgggcatgaccagcgcgctggcgacccgcattgcgaacgaactgaacgaagtgctggtgggcgtgaacctgccgattaacgcgtgctatattcatgtggatctgccgagcctgagcaacgcggtgtatgcgaaactggcgcatctgaaactgccggatcgcaccccggaaccgcgcaaagcgccggtggaaaacccgggcggcaaagaaattgtgattgtgggccaggcgtttcgcctgccgggcagcattaacgatgtggcgagcctgcgcgatgcgtttctggcgcgccaggcgagcagcattattaccgaaattccgccggatcgctgggatcatgcgagcttttatccgaaagatattcgctttaacaaagcgggcctggtggatattgcgaactatgatcatagcttttttggcctgaccgcgaccgaagcgctgtatctgagcccgaccatgcgcctggcgctggaagtgagctttgaagcgctggaaaacgcgaacattccggtgagccagctgaaaggcagccagaccgcggtgtatgtggcgaccaccgatgatggctttgaaaccctgctgaacgcggaagcgggctatgatgcgtatacccgcttttatggcaccggccgcgcggcgagcaccgcgagcggccgcattagctatctgctggatgtgcatggcccgagcattaccgtggataccgcgtgcagcggcggcgcggtgtgcattgatcaggcgattgattatctgcagagcagcagcgcggcggataccgcgattatttgcgcgagcaacacccattgctggccgggcagctttatgtttctgagcgcgcagggcatggtgagcagcggcggccgctgcgcgacctttaccaccgatgcggatggctatgtgccgagcgaaggcgcggtggcgtttattctgaaaacccgcgaagcggcgatgcgcgataaagataccattctggcgaccattaaagcgacccagattagccataacggccgcagccagggcctggtggcgccgaacgtgaacagccaggcggatctgcatcgcagcctgctgcagaaagcgggcctgagcccggcggatattcattttattgaagcgcatggcaccggcaccagcctgggcgatctgagcgaaattcaggcgattaacgatgcgtataccagcagccagccgcgcaccgcgggcccgctgattgtgagcgcgagcaaaaccgtgattggccataccgaaccggcgggcccgctggtgggcatgctgagcgtgctgaacagctttaaagaaggcgcggtgccgggcctggcgcatctgaccgcggataacctgaacccggcgctggattgcagcagcgtgccgctgctgattccgtatcagccggtgcatctggcggcgccgaaaccgcatcgcgcggcggtgatgagctatggctttagcggcaccctgggcggcattgtgctggaagcgccggatgaagaacgcctggaagaagaaccgccgaacgataaaccgatgctgtttgtggtgagcgcgaaaacccataccgcgctgattgaatatctgggccgctatctggaatttctgctgcaggcgaacccgcaggatttttgcgatatttgctataccagctgcgtgggccgcgaacattatcgctatcgctttgcgtgcgtggcgaacgatatggaagatctgattggccagctgcagaaacgcctgggcagcaaagtgccgccgaaaccgagctataaacgcggcgcgctggcgtttgcgtttagcggccagggcacccagtttcgcggcatggcgaccgaactggcgaaagcgtatagcggctttcgcaaaattgtgagcgatctggcgaaacgcgcgagcgaactgagcggccatgcgattgatcgctttctgctggcgtatgatattggcgcggaaaacgtggcgccggatagcgaagcggatcagatttgcatttttgtgtatcagtgcagcgtgctgcgctggctgcagaccatgggcattcgcccgagcgcggtgattggccatagcctgggcgaaattagcgcgagcgtggcggcgggcgcgctgagcctggatagcgcgctggatctggtgattagccgcgcgcgcctgctgcgcagcagcaccaacgcgccggcgggcatggcggcgatgagcgcgagccaggatgaagtggtggaactgattggcaaactggatctggataaagcgaacagcctgagcgtgagcgtgattaacggcccgcagaacaccgtggtgagcggcagcagcgcggcgattgaaagcattgtggcgctggcgaaaggccgcaaaattaaagcgagcgcgctgaacattaaccaggcgtttcatagcccgtatgtggatagcgcggtgccgggcctgcgcgcgtggagcgaaaaacatattagcagcgcgcgcccgctgcagattccgctgtatagcaccctgctgggcgcgcaggtgagcgaaggccagatgctgaacccggatcattgggtggatcatgcgcgcaaaccggtgcagtttgcgcaggcggcgaccattatgaaagaaagctttaccggcgtgattattgatattggcccgcaggtggtggcgtggagcctgctgctgagcaacggcctgaccagcgtgaccgcgctggcggcgaaacgcggccgcagccagcaggtggcgtttctgagcgcgctggcggatctgtatcaggattatggcgtggtgccggattttgtgggcctgtatgcgcagcaggaagatgcgagccgcctgaaaaaaaccgatattctgacctatccgtttcagcgcgtgcgccgctatccgagctttattccgagccgccgcgcgccgacccatgcgcatgtgcaggatgaagaaaccctgagcagcggcagcagcaccccgaccctggaaaacaccgatctggatagcggcaaagaaagcctgatgggcccgacccgcggcctgctgcgcgtggatgatctgcgcgatagcattgtgagcagcgtgaaagatgtgctggaactgaaaagcaacgaagatctggatctgagcgaaagcctgaacgcgctgggcatggatagcattatgtttgcgcagctgcgcaaacgcattggcgaaggcctgggcctgagcgtgccgatggtgtttctgagcgatgcgtttagcattggcgaaatggtgagcaacctggtggaacaggcggaagcgagcgaagataac

SEQ ID NO: 48 - DNA, 714 bp, Artificial Sequence
  Synthetic construct
atgagcaaaggcgaagaactgtttaccggcgtggtgccgattctggtggaactggatggcgatgtgaacggccataaatttagcgtgagcggcgaaggcgaaggcgatgcgacctatggcaaactgaccctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccattagctttaaagatgatggcaactataaaacccgcgcggaagtgaaatttgaaggcgataccctggtgaaccgcattgaactgaaaggcattgattttaaagaagatggcaacattctgggccataaactggaatataactataacagccataacgtgtatattaccgcggataaacagaaaaacggcattaaagcgaactttaaaacccgccataacattgaagatggcggcgtgcagctggcggatcattatcagcagaacaccccgattggcgatggcccggtgctgctgccggataaccattatctgagcacccagagcgcgctgagcaaagatccgaacgaaaaacgcgatcatatggtgctgctggaatttgtgaccgcggcgggcattacccatggcatggatgaactgtataaa

SEQ ID NO: 49 - DNA, 717 bp, Artificial Sequence
  Cloning vector pCI-YFP
atggtgagcaaaggcgaagaactgtttaccggcgtggtgccgattctggtggaactggatggcgatgtgaacggccataaatttagcgtgagcggcgaaggcgaaggcgatgcgacctatggcaaactgaccctgaaatttatttgcaccaccggcaaactgccggtgccgtggccgaccctggtgaccacctttggctatggcctgcagtgctttgcgcgctatccggatcatatgaaactgcatgatttttttaaaagcgcgatgccggaaggctatgtgcaggaacgcaccattttttttaaagatgatggcaactataaaacccgcgcggaagtgaaatttgaaggcgataccctggtgaaccgcattgaactgaaaggcattgattttaaagaagatggcaacattctgggccataaactggaatataactataacagccataacgtgtatattatggcggataaacagaaaaacggcattaaagtgaactttaaaattcgccataacattgaagatggcagcgtgcagctggcggatcattatcagcagaacaccccgattggcgatggcccggtgctgctgccggataaccattatctgagctatcagagcgcgctgagcaaagatccgaacgaaaaacgcgatcatatggtgctgctggaatttgtgaccgcggcgggcattaccctgggcatggatgaactgtataaa

SEQ ID NO: 50 - DNA, 675 bp, Artificial Sequence
  Cloning vector pHUGE-RedSeed
atgcgcagcagcaaaaacgtgattaaagaatttatgcgctttaaagtgcgcatggaaggcaccgtgaacggccatgaatttgaaattgaaggcgaaggcgaaggccgcccgtatgaaggccataacaccgtgaaactgaaagtgaccaaaggcggcccgctgccgtttgcgtgggatattctgagcccgcagtttcagtatggcagcaaagtgtatgtgaaacatccggcggatattccggattataaaaaactgagctttccggaaggctttaaatgggaacgcgtgatgaactttgaagatggcggcgtggtgaccgtgacccaggatagcagcctgcaggatggctgctttatttataaagtgaaatttattggcgtgaactttccgagcgatggcccggtgatgcagaaaaaaaccatgggctgggaagcgagcaccgaacgcctgtatccgcgcgatggcgtgctgaaaggcgaaattcataaagcgctgaaactgaaagatggcggccattatctggtggaatttaaaagcatttatatggcgaaaaaaccggtgcagctgccgggctattattatgtggatagcaaactggatattaccagccataacgaagattataccattgtggaacagtatgaacgcaccgaaggccgccatcatctgtttctg

SEQ ID NO: 51 - DNA, 675 bp, Artificial Sequence
  Cloning vector OX4509
atggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctg

SEQ ID NO: 52 - DNA, 708 bp, Artificial Sequence
  Synthetic construct
atggtgagcaaaggcgaagaaaacaacatggcgattattaaagaatttatgcgctttaaagtgcgcatggaaggcagcgtgaacggccatgaatttgaaattgaaggcgaaggcgaaggccgcccgtatgaaggctttcagaccgcgaaactgaaagtgaccaaaggcggcccgctgccgtttgcgtgggatattctgagcccgcagtttacctatggcagcaaagcgtatgtgaaacatccggcggatattccggattattttaaactgagctttccggaaggctttaaatgggaacgcgtgatgaactttgaagatggcggcgtggtgaccgtgacccaggatagcagcctgcaggatggcgaatttatttataaagtgaaactgcgcggcaccaactttccgagcgatggcccggtgatgcagaaaaaaaccatgggctgggaagcgagcagcgaacgcatgtatccggaagatggcgcgctgaaaggcgaaattaaaatgcgcctgaaactgaaagatggcggccattataccagcgaagtgaaaaccacctataaagcgaaaaaaccggtgcagctgccgggcgcgtatattgtgggcattaaactggatattaccagccataacgaagattataccattgtggaacagtatgaacgcgcggaaggccgccatagcaccggcggcatggatgaactgtataaa

SEQ ID NO: 53 - DNA, 708 bp, Artificial Sequence
  Cloning vector pNIGEL17
atgctgagcaaaggcgaagaagataacatggcgattattaaagaatttatgcgctttaaagtgcatatggaaggcagcgtgaacggccatgaatttgaaattgaaggcgaaggcgaaggccgcccgtatgaaggcacccagaccgcgaaactgaaagtgaccaaaggcggcccgctgccgtttgcgtgggatattctgagcccgcagtttatgtatggcagcaaagcgtatgtgaaacatccggcggatattccggattatctgaaactgagctttccggaaggctttaaatgggaacgcgtgatgaactttgaagatggcggcgtggtgaccgtgacccaggatagcagcctgcaggatggcgaatttatttataaagtgaaactgcgcggcaccaactttccgagcgatggcccggtgatgcagaaaaaaaccatgggctgggaagcgagcagcgaacgcatgtatccggaagatggcgcgctgaaaggcgaaattaaacagcgcctgaaactgaaagatggcggccattatgatgcggaagtgaaaaccacctataaagcgaaaaaaccggtgcagctgccgggcgcgtataacgtgaacattaaactggatattaccagccataacgaagattataccattgtggaacagtatgaacgcgcggaaggccgccatagcaccggcggcatggatgaactgtataaa

SEQ ID NO: 54 - DNA, 1080 bp, Homo sapiens
atggcgggccatctggcgagcgattttgcgtttagcccgccgccgggcggcggcggcgatggcccgggcggcccggaaccgggctgggtggatccgcgcacctggctgagctttcagggcccgccgggcggcccgggcattggcccgggcgtgggcccgggcagcgaagtgtggggcattccgccgtgcccgccgccgtatgaattttgcggcggcatggcgtattgcggcccgcaggtgggcgtgggcctggtgccgcagggcggcctggaaaccagccagccggaaggcgaagcgggcgtgggcgtggaaagcaacagcgatggcgcgagcccggaaccgtgcaccgtgaccccgggcgcggtgaaactggaaaaagaaaaactggaacagaacccggaagaaagccaggatattaaagcgctgcagaaagaactggaacagtttgcgaaactgctgaaacagaaacgcattaccctgggctatacccaggcggatgtgggcctgaccctgggcgtgctgtttggcaaagtgtttagccagaccaccatttgccgctttgaagcgctgcagctgagctttaaaaacatgtgcaaactgcgcccgctgctgcagaaatgggtggaagaagcggataacaacgaaaacctgcaggaaatttgcaaagcggaaaccctggtgcaggcgcgcaaacgcaaacgcaccagcattgaaaaccgcgtgcgcggcaacctggaaaacctgtttctgcagtgcccgaaaccgaccctgcagcagattagccatattgcgcagcagctgggcctggaaaaagatgtggtgcgcgtgtggttttgcaaccgccgccagaaaggcaaacgcagcagcagcgattatgcgcagcgcgaagattttgaagcggcgggcagcccgtttagcggcggcccggtgagctttccgctggcgccgggcccgcattttggcaccccgggctatggcagcccgcattttaccgcgctgtatagcagcgtgccgtttccggaaggcgaagcgtttccgccggtgagcgtgaccaccctgggcagcccgatgcatagcaac

SEQ ID NO: 55 - DNA, 951 bp, Homo sapiens
atgtataacatgatggaaaccgaactgaaaccgccgggcccgcagcagaccagcggcggcggcggcggcaacagcaccgcggcggcggcgggcggcaaccagaaaaacagcccggatcgcgtgaaacgcccgatgaacgcgtttatggtgtggagccgcggccagcgccgcaaaatggcgcaggaaaacccgaaaatgcataacagcgaaattagcaaacgcctgggcgcggaatggaaactgctgagcgaaaccgaaaaacgcccgtttattgatgaagcgaaacgcctgcgcgcgctgcatatgaaagaacatccggattataaatatcgcccgcgccgcaaaaccaaaaccctgatgaaaaaagataaatataccctgccgggcggcctgctggcgccgggcggcaacagcatggcgagcggcgtgggcgtgggcgcgggcctgggcgcgggcgtgaaccagcgcatggatagctatgcgcatatgaacggctggagcaacggcagctatagcatgatgcaggatcagctgggctatccgcagcatccgggcctgaacgcgcatggcgcggcgcagatgcagccgatgcatcgctatgatgtgagcgcgctgcagtataacagcatgaccagcagccagacctatatgaacggcagcccgacctatagcatgagctatagccagcagggcaccccgggcatggcgctgggcagcatgggcagcgtggtgaaaagcgaagcgagcagcagcccgccggtggtgaccagcagcagccatagccgcgcgccgtgccaggcgggcgatctgcgcgatatgattagcatgtatctgccgggcgcggaagtgccggaaccggcggcgccgagccgcctgcatatgagccagcattatcagagcggcccggtgccgggcaccgcgattaacggcaccctgccgctgagccatatg

SEQ ID NO: 56 - DNA, 1539 bp, Homo sapiens
atgcgccagccgccgggcgaaagcgatatggcggtgagcgatgcgctgctgccgagctttagcacctttgcgagcggcccggcgggccgcgaaaaaaccctgcgccaggcgggcgcgccgaacaaccgctggcgcgaagaactgagccatatgaaacgcctgccgccggtgctgccgggccgcccgtatgatctggcggcggcgaccgtggcgaccgatctggaaagcggcggcgcgggcgcggcgtgcggcggcagcaacctggcgccgctgccgcgccgcgaaaccgaagaatttaacgatctgctggatctggattttattctgagcaacagcctgacccatccgccggaaagcgtggcggcgaccgtgagcagcagcgcgagcgcgagcagcagcagcagcccgagcagcagcggcccggcgagcgcgccgagcacctgcagctttacctatccgattcgcgcgggcaacgatccgggcgtggcgccgggcggcaccggcggcggcctgctgtatggccgcgaaagcgcgccgccgccgaccgcgccgtttaacctggcggatattaacgatgtgagcccgagcggcggctttgtggcggaactgctgcgcccggaactggatccggtgtatattccgccgcagcagccgcagccgccgggcggcggcctgatgggcaaatttgtgctgaaagcgagcctgagcgcgccgggcagcgaatatggcagcccgagcgtgattagcgtgagcaaaggcagcccggatggcagccatccggtggtggtggcgccgtataacggcggcccgccgcgcacctgcccgaaaattaaacaggaagcggtgagcagctgcacccatctgggcgcgggcccgccgctgagcaacggccatcgcccggcggcgcatgattttccgctgggccgccagctgccgagccgcaccaccccgaccctgggcctggaagaagtgctgagcagccgcgattgccatccggcgctgccgctgccgccgggctttcatccgcatccgggcccgaactatccgagctttctgccggatcagatgcagccgcaggtgccgccgctgcattatcagggccagagccgcggctttgtggcgcgcgcgggcgaaccgtgcgtgtgctggccgcattttggcacccatggcatgatgctgaccccgccgagcagcccgctggaactgatgccgccgggcagctgcatgccggaagaaccgaaaccgaaacgcggccgccgcagctggccgcgcaaacgcaccgcgacccatacctgcgattatgcgggctgcggcaaaacctataccaaaagcagccatctgaaagcgcatctgcgcacccataccggcgaaaaaccgtatcattgcgattgggatggctgcggctggaaatttgcgcgcagcgatgaactgacccgccattatcgcaaacataccggccatcgcccgtttcagtgccagaaatgcgatcgcgcgtttagccgcagcgatcatctggcgctgcatatgaaacgccatttt

SEQ ID NO: 57 - DNA, 1313 bp, Mus musculus
atgccgctgaacgtgaactttaccaaccgcaactatgatctggattatgatagcgtgcagccgtattttatttgcgatgaagaagaaaacttttatcatcagcagcagcagagcgaactgcagccgccggcgccgagcgaagatatttggaaaaaatttgaactgctgccgaccccgccgctgagcccgagccgccgcagcggcctgtgcagcccgagctatgtggcggtggcgaccagctttagcccgcgcgaagatgatgatggcggcggcggcaactttagcaccgcggatcagctggaaatgatgaccgaactgctgggcggcgatatggtgaaccagagctttatttgcgatccggatgatgaaacctttattaaaaacattattattcaggattgcatgtggagcggctttagcgcggcggcgaaactggtgagcgaaaaactggcgagctatcaggcggcgcgcaaagatagcaccagcctgagcccggcgcgcggccatagcgtgtgcagcaccagcagcctgtatctgcaggatctgaccgcggcggcgagcgaatgcattgatccgagcgtggtgtttccgtatccgctgaacgatagcagcagcccgaaaagctgcaccagcagcgatagcaccgcgtttagcccgagcagcgatagcctgctgagcagcgaaagcagcccgcgcgcgagcccggaaccgctggtgctgcatgaagaaaccccgccgaccaccagcagcgatagcgaagaagaacaggaagatgaagaagaaattgatgtggtgagcgtggaaaaacgccagaccccggcgaaacgcagcgaaagcggcagcagcccgagccgcggcatagcaaaccgccgcatagcccgctggtgctgaaacgctgccatgtgagcacccatcagcataactatgcggcgccgccgagcacccgcaaagattatccggcggcgaaacgcgcgaaactggatagcggccgcgtgctgaaacagattagcaacaaccgcaaatgcagcagcccgcgcagcagcgataccgaagaaaacgataaacgccgcacccataacgtgctggaacgccagcgccgcaacgaactgaaacgcagcttttttgcgctgcgcgatcagattccggaactggaaaacaacgaaaaagcgccgaaagtggtgattctgaaaaaagcgaccgcgtatattctgagcattcaggcggatgaacataaactgaccagcgaaaaagatctgctgcgcaaacgccgcgaacagctgaaacataaactggaacagctgcgcaacagcggc

SEQ ID NO: 58 - DNA, 12 bp, Artificial Sequence
  Furin recognition sequence
cggaagcggaga

SEQ ID NO: 59 - DNA, 9 bp, Artificial Sequence
  peroxisome targeting sequence
agcaagctg

SEQ ID NO: 60 - DNA, 54 bp, Artificial Sequence
  T2a linker sequence
gagggccgcggcagcctgctgacctgcggcgacgtggaggagaaccccggcccc

SEQ ID NO: 61 - DNA, 60 bp, Artificial Sequence
  E2a linker seuqence
cagtgcaccaactacgccctgctgaagctggccggcgacgtggagagcaaccccggcccc

SEQ ID NO: 62 - DNA, 57 bp, Artificial Sequence
  P2a linker sequence
gcgacaaactttagcttgctgaagcaagctggtgacgttgaggagaatcccggacca

SEQ ID NO: 63 - DNA, 57 bp, Artificial Sequence
  Pa2a linker sequence
gccaccaacttcagcctgctgaagcaggccggcgacgtggaggagaaccccggcccc

SEQ ID NO: 64 - DNA, 60 bp, Artificial Sequence
  FMDV2a linker sequence
cagctgctgaacttcgacctgctgaagctggccggcgacgtggagagcaaccccggcccc

SEQ ID NO: 65 - protein, 306 aa, Artificial Sequence
  Cloning vector pX037
MAPISSTWSRLIRFVAVETSLVHIGEPIDATMDVGLARREGKTIQAYEIIGSGSALDLSAQVSKNVLTVRELLMPLSREEIKTVRCLGLNYPVHATEANVAVPKFPNLFYKPVTSLIGPGGLITIPSVVQPPKEHQSDYEAELVIVIGKAAKNVSEDEALDYVLGYTAANDISFRKHQLAVSQWSFSKGFDGTNPLGPCLVSASSIPDPQDIPIQCKLNGGVVQNGNTRDQIFNVKKTISFLSQGTTLEPGSIILTGTPDGVGFVRNPPLYLKDGDEVMTWIGSGIGTLANTVREEQTCFASGGHE

SEQ ID NO: 66 - protein, 346 aa, Artificial Sequence
  Cloning vector pnpgA-C1
MVQDTSSASTSPILTRWYIDTRPLTASTAALPLLETLQPADQISVQKYYHLKDKHMSLASNLLKYLFVHRNCRIPWSSIVISRTPDPHRRPCYIPPSGSQEDSFKDGYTGINVEFNVSHQASMVAIAGTAFTPNSGGDSKLKPEVGIDITCVNERQGRNGEERSLESLRQYIDIFSEVFSTAEMANIRRLDGVSSSSLSADRLVDYGYRLFYTYWALKEAYIKMTGEALLAPWLRELEFSNVVAPAAVAESGDSAGDFGEPYTGVRTTLYKNLVEDVRIEVAALGGDYLFATAARGGGIGASSRPGGGPDGSGIRSQDPWRPFKKLDIERDIQPCATGVCNCLSRG

SEQ ID NO: 67 - protein, 533 aa, Artificial Sequence
  Cloning vector pRcTAL-C1
MTLQSQTAKDCLALDGALTLVQCEAIATHRSRISVTPALRERCARAHARLEHAIAEQRHIYGITTGFGPLANRLIGADQGAELQQNLIYHLATGVGPKLSWAEARALMLARLNSILQGASGASPETIDRIVAVLNAGFAPEVPAQGTVGASGDLTPLAHMVLALQGRGRMIDPSGRVQEAGAVMDRLCGGPLTLAARDGLALVNGTSAMTAIAALTGVEAARAIDAALRHSAVLMEVLSGHAEAWHPAFAELRPHPGQLRATERLAQALDGAGRVCRTLTAARRLTAADLRPEDHPAQDAYSLRVVPQLVGAVWDTLDWHDRVVTCELNSVTDNPIFPEGCAVPALHGGNFMGVHVALASDALNAALVTLAGLVERQIARLTDEKLNKGLPAFLHGGQAGLQSGFMGAQVTATALLAEMRANATPVSVQSLSTNGANQDVVSMGTIAARRARAQLLPLSQIQAILALALAQAMDLLDDPEGQAGWSLTARDLRDRIRAVSPGLRADRPLAGHIEAVAQGLRHPSAAADPPASG

SEQ ID NO: 68 - protein, 522 aa, Artificial Sequence
  Cloning vector pHpaB-C1
MKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQNSGNMDKMMAMVDRCLSEYDQDGWTVPHLHNNDDINMLDKLLKSG

SEQ ID NO: 69 - protein, 172 aa, Artificial Sequence
  Cloning vector pHpaC-C1
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAISG

SEQ ID NO: 70 - DNA, 921 bp, Artificial Sequence
  Cloning vector pX037
atggcacctatttcatccacgtggagcaggttgataaggttcgtggctgtagaaacttccctggttcatattggtgagccaattgatgcaacgatggacgttggcctcgcccgtagagaaggtaagacaattcaagcatacgaaataatcggaagcggcagtgccctcgatctttctgcacaagtctctaagaacgtactcacagttagggaattgttgatgccgctcagtcgagaagagattaagaccgttcgatgcctcggcttgaactatccggtccacgccacagaagctaacgtggccgtgccgaagtttcctaacctgttctataagcccgtaacatcattgattggtccaggcggtcttattactattccctccgtcgttcaaccccccaaggagcaccagtcagattacgaggctgagctggttatagtaataggtaaggcagcaaagaacgtatcagaggatgaggctttggattatgtattgggctataccgctgcaaacgatatatctttccgtaagcatcaattggctgtcagtcaatggagcttcagcaagggctttgatggaactaatccacttggtccctgcttggtttccgcctccagtatccctgacccacaggatattccaatacagtgcaaattgaatggtggagtcgtccaaaatggcaacaccagagatcaaatcttcaacgttaagaaaaccatatcatttctttcccaggggactactttggagcccggttccattatccttaccgggaccccagatggggtggggttcgtacgaaacccgcccctgtatttgaaggatggcgatgaggttatgacttggatcggtagtggaatcggaactctcgcaaatacagttcgagaagaacaaacgtgctttgcatcaggcggccacgaatag

SEQ ID NO: 71 - DNA, 1040 bp, Artificial Sequence
  Cloning vector pnpgA-C1
tggtccaagacacctcaagcgctagtaccagtccaattttgacaagatggtatatagatactagacctttgactgcttctaccgcagccttgccactgttggaaactctgcagcctgcagaccagataagtgtccaaaagtactaccatctgaaggataagcatatgtctctcgcatccaatcttctcaagtacttgtttgttcacaggaattgccgaatcccatggtctagcattgtaatatcaagaactccagatccccatagaaggccctgttatataccaccctctggttctcaagaggacagctttaaggatggctacactggaattaatgtcgagttcaacgtatcccatcaagcaagcatggtggcaatagcaggcactgcatttacacccaactctggaggtgattccaaacttaagccagaggttggcatagacattacttgcgtgaatgagcgacaggggcgaaacggtgaggagcgatcccttgaaagtttgaggcagtacatagatattttctcagaggttttttccaccgcagagatggcaaacatcagaagattggatggggtgtcatcaagctccttgagcgccgataggttggtggactatggttaccgattgttttacacttattgggctcttaaggaagcatacatcaagatgacaggggaagccctgctggcaccatggctcagggagttggaatttagtaatgtcgttgccccagcagcagtggccgaatccggagattccgccggagactttggagagccttataccggggtacgaacaaccctttacaaaaacttggtcgaggatgttaggatagaggtggctgctctgggaggtgactacttgttcgccactgctgcacgagggggcggtataggtgcctccagccgaccagggggaggccctgatggctcaggaattaggagtcaagatccctggcgaccatttaaaaagctcgacatagagagagacatacagccttgcgccactggagtctgtaattgtctgtctcgaggttga

SEQ ID NO: 72 - DNA, 1602 bp, Artificial Sequence
  Cloning vector pRcTAL-C1
atgactcttcaatcccaaactgctaaggattgcttggcactggatggtgcactgactttggtgcagtgcgaggcaatcgccacgcatcgtagtcgtatttccgttacgccagccttgcgtgaacgttgtgctcgtgcccacgcacgtttggagcacgcaatcgcagagcagcgtcatatttacggtatcaccactggatttggacccctggctaaccgtcttattggagccgatcagggcgctgagctgcaacagaatctgatttaccatcttgcaacgggcgttggacccaagctgtcctgggcagaggcccgtgcacttatgttggcacgtcttaattccattttgcagggcgctagtggtgcttctcctgaaacgatcgaccgtatcgtcgcagtacttaacgctggctttgctcctgaagttcccgcacaaggtactgttggcgcaagtggcgacttgactccccttgcccacatggtactggccctgcaaggccgtggacgtatgatcgatcctagtggacgtgtgcaagaggctggagccgtcatggatcgtctttgcggaggccctcttacgttggccgcacgtgacggccttgctctggtcaacggtacaagtgctatgactgctatcgctgctcttactggagtcgaggccgctcgtgctatcgatgctgcacttcgtcactccgccgttttgatggaagtcctgtctggacatgcagaagcctggcaccccgcctttgcagagttgagaccccacccaggccagttgcgtgctactgaacgtttggcccaggccctggatggcgcaggccgtgtatgccgtactctgacggctgcccgtcgtcttactgcagccgacctgcgtccagaggaccaccccgcacaagacgcttattctctgcgtgtcgttcctcaactggttggtgccgtatgggatactcttgactggcacgaccgtgtcgtgacctgtgagcttaattcagtaaccgataaccccatttttccagagggttgcgccgttcctgcacttcacggaggaaacttcatgggagtccacgtcgcactggcttcagacgctttgaatgcagccctggttacccttgccggtcttgtagagcgtcaaattgcccgtcttaccgacgagaaacttaataaaggtctgcctgctttccttcacggtggacaggcaggccttcagagtggcttcatgggcgctcaggttacagcaactgccctgttggctgagatgcgtgcaaatgcaactccagtctcagtacaatccttgtccacaaacggtgcaaaccaagacgtcgtatctatgggaactatcgccgcacgtcgtgctcgtgcacaattgttgccactgtcacagattcaggctatcttggctttggcattggcccaggctatggatcttcttgacgatcccgagggtcaagccggatggtctttgacagcccgtgatttgcgtgaccgtatccgtgccgtttctcccggactgcgtgccgaccgtccactggcaggccatatcgaagctgttgctcagggacttcgtcacccatctgctgcagccgatccaccagcctcaggttga

SEQ ID NO: 73 - DNA, 1569 bp, Artificial Sequence
  Cloning vector pHpaB-C1
atgaaacctgaagattttcgtgcttccacccagcggccctttactggagaagaatatctgaagtctcttcaagacggccgtgagatctatatttacggcgagcgtgtaaaggatgttacaacgcaccctgcttttcgtaatgctgctgcctcagttgcccaactttatgacgccctgcataaacccgaaatgcaggactcactttgttggaatacggacacggggtccggtggttacacacacaagttctttcgtgtcgccaaatctgccgatgacttgcgtcaacaacgtgatgctatcgctgaatggtcacgtctgagttacggctggatgggccgtacgcccgactataaggccgcctttggttgcgcattgggtgcaaatcccggcttttatggacagtttgaacaaaatgcacgtaactggtatactcgtatccaggaaaccggcttgtactttaatcatgctattgttaacccacctatcgatcgtcatttgccaaccgataaggtcaaggatgtatatattaaacttgaaaaggagacggacgctggtatcattgtgtctggcgctaaagtggtggctacgaacagtgcattgacgcattacaatatgattggttttggttccgcccaagtgatgggcgagaaccccgactttgccttgatgtttgttgcccctatggacgctgacggagtaaaacttatttcacgtgcctcatatgaaatggtagctggtgccactggttctccttatgattatcctctttcatcccgttttgacgaaaacgacgccatcctggtcatggacaatgttcttattccctgggagaacgtgctgatctaccgtgacttcgatcgttgccgtcgttggactatggaaggaggtttcgctcgtatgtatccactgcaggcctgtgttcgtttggctgtgaagttggactttatcacagcccttcttaaaaagtcacttgagtgtactggaacgttggagttccgtggtgtacaggcagatctgggcgaagtagtagcctggcgtaatactttctgggcactgtctgactctatgtgctccgaggcaacaccatgggtgaacggcgcatacctgcccgatcacgccgctcttcaaacatatcgtgtgcttgctcccatggcttatgctaaaatcaaaaatattatcgagcgtaatgtaacttctggtctgatttacctgccatccagtgctcgtgaccttaacaatcctcaaatcgatcagtatcttgcaaaatatgtgcgtggctcaaacggaatggaccatgtgcagcgtatcaaaatcctgaagttgatgtgggatgccatcggttctgaatttggcggacgtcatgaactgtatgagattaactactctggatctcaagacgaaatcagacttcagtgtttgcgtcaggcccaaaattctggaaatatggataaaatgatggctatggtagaccgttgtctgagtgagtacgatcaagatggttggacagttcctcatcttcataataatgacgatattaatatgcttgataagttgcttaaatcaggttga

SEQ ID NO: 74 - DNA, 519 bp, Artificial Sequence
  Cloning vector pHpaC-C1
atgcagcttgatgagcagcgtttgcgttttcgtgacgctatggcatcacttagtgccgctgtgaacatcatcacgactgagggagacgccggtcagtgcggaatcacagcaaccgcagtatgtagtgttacggacactcccccatctctgatggtctgtatcaatgccaatagtgctatgaatccagtctttcagggtaatggtaaactttgtgtcaacgtcctgaatcacgagcaagagttgatggctcgtcattttgcaggaatgactggtatggctatggaagagcgtttttccttgtcttgttggcaaaaaggaccactggcccagcctgtattgaaaggttctttggcttccctggagggtgagatccgtgacgttcaagccattggaactcatctggtatatttggtagaaatcaagaatattatcctgtcagccgagggccatggtctgatctactttaaacgtcgttttcaccccgtgatgctggagatggaggcagccatttcaggttga